(19) United States
(12) Patent Application Publication
(10) Pub. No.: US 2026/0158144 A1
(43) Pub. Date: Jun. 11, 2026

(54) PHOTODYNAMIC THERAPY ILLUMINATOR DEVICES AND METHODS

(71) Applicant: Sun Pharmaceutical Industries, Inc., Princeton, NJ (US)

(72) Inventors: Thomas BOYAJIAN, Wilmington, MA (US); Mark CAROTA, Chelmsford, MA (US); Brian MAZEJKA, Salem, NH (US); Pradeep SANGHVI, Brampton (CA); Scott LUNDAHL, Billerica, MA (US); Xiaopin JIN, Mississauga (CA); Brenden B. HADJIKEZIAN, Vaughan (CA); Jerzy ZADYKOWICZ, Mississauga (CA); Haydar ABDALGHAFOR, Brampton (CA); Tasnuva HAQUE, Brampton (CA)

(73) Assignee: Sun Pharmaceutical Industries, Inc., Princeton, NJ (US)

(21) Appl. No.: 18/707,320

(22) PCT Filed: Nov. 4, 2022
(86) PCT No.: PCT/IB2022/060654

§ 371 Date: May 3, 2024

Related U.S. Application Data
(60) Provisional application No. 63/276,312, filed on Nov. 5, 2021.

(51) Int. Cl. A61K 41/00; A61B 5/00; A61K 9/00; A61K 47/18; A61N 5/00; A61N 5/06

(52) U.S. Cl. CPC A61K 41/0061; A61B 5/441; A61K 9/0014; A61K 47/183; A61N 5/062; A61N 2005/007; A61N 2005/0633

(57) ABSTRACT

A method of performing photodynamic therapy is provided. The method includes applying, to the skin of a patient, a topical composition. The topical composition includes 5-aminolevulinic acid (ALA) hydrochloride, and a vehicle comprising at least one chelating agent to enhance accumulation of protoporphyrin IX (PpIX) in the skin. The method further includes incubating the topical composition, and following incubation, applying, to the skin, heat from a heat source for at least a first time period.

### CROSS-REFERENCE TO RELATED APPLICATION(S)

The present application claims priority to U.S. Provisional Application No. 63/276,312 filed Nov. 5, 2021, the entire contents of which are incorporated herein by reference.

### FIELD

The present disclosure relates generally to devices and methods for photodynamic therapy.

### BACKGROUND

Photodynamic therapy (PDT), photodynamic diagnosis (PD), and/or photochemotherapy generally involve treating and/or diagnosing several types of diseases or disorders relating to the skin or other tissues, such as those in a body cavity. PDT can include administering photoactivatable agents and exposing a patient to photoactivating light to activate the agents and convert them to cytotoxic form, to destroy cells associated with a disease or disorder of the skin. For example, photodynamic therapy or photodynamic diagnosis may be used for treatment or diagnosis of actinic keratosis (AK) of the upper extremities (e.g., the dorsal surface of the hand or forearms), the scalp or facial areas of a patient, among other locations. AK is typically caused by overexposure to ultraviolet (UV) light and may present on the face, head, scalp, ears, shoulders, neck, arms, forearms and hands, for example.

In addition, PDT or PD may be used for treatment and diagnosis of other indications (e.g., acne, warts, psoriasis, photo-damaged skin, cancer) and other areas of the patient (e.g., the legs or portions of the arms other than the forearms, the back, the abdomen, the chest, or another portion of a body). PDT using UV or blue light is indicated for the treatment of mild to moderate acne, due to anti-inflammatory effects on skin cells. The combination of a photoactivatable agent and high intensity red light has been found effective, yet can have significant side effects.

During a form of photodynamic therapy, a patient is first administered a photoactivatable agent or a precursor of a photoactivatable agent that accumulates in the tissue to be treated. The area to which the photoactivatable agent is administered is then exposed to light, which causes chemical and/or biological changes in the agent. These changes allow the agent to then selectively locate, destroy, or alter the target tissue while, at the same time, causing at most only mild and reversible damage to other tissues in the treatment area. One example of a precursor of a photoactivatable agent is 5-aminolevulinic acid (“ALA” or “5-ALA”), which is commonly used in photodynamic therapy of actinic keratosis. As used herein, the terms ALA or 5-aminolevulinic acid refer to ALA itself, precursors thereof, esters thereof and pharmaceutically acceptable salts of the same, such as aminolevulinic acid hydrochloride (HCl). Photosensitization following application of a topical composition (e.g., a topical solution, an emulsion, a nanoemulsion, a gel) containing ALA occurs through the metabolic conversion of aminolevulinic acid to protoporphyrin IX (“PpIX”). PpIX is a photosensitizer which accumulates in the skin.

Illuminators are typically used to provide the proper uniformity of light for treatment purposes. These devices generally include a light source (e.g., a fluorescent tube or light emitting diode (LED)), coupling elements that direct, filter or otherwise conduct emitted light so that it arrives at its intended target in a usable form, and a control system that starts and stops the production of light when necessary.

Photodynamic therapy may be carried out using certain compositions, such as ALA, in connection with illuminators. Such compositions and/or illuminators (as well as methods of treatment, dressings, and other details) are disclosed, for example, in (1) U.S. Pat. No. 5,954,703 to Golub, entitled “Method and apparatus for applying 5-aminolevulinic acid,” issued on Sep. 21, 1999, (2) U.S. Pat. No. 6,223,071 to Lundahl et al., entitled “Illuminator for photodynamic therapy and diagnosis which produces substantially uniform intensity visible light,” issued on Apr. 24, 2001, (3) U.S. Pat. No. 10,814,114 to Boyajian et al., entitled “Method and apparatus for applying a topical solution,” issued on Oct. 27, 2020, (4) U.S. Pat. No. 10,589,122 to Boyajian et al., entitled “Adjustable illuminator for photodynamic therapy and diagnosis,” issued on Mar. 17, 2020, (5) U.S. Patent Application Publication No. 2020/0246630 to Boyajian et al, entitled “Adjustable illuminator for photodynamic therapy and diagnosis,” published on Aug. 6, 2020, (6) U.S. Pat. No. 11,179,574 to Boyajian et al., entitled “Method of administering 5-aminolevulinic acid (ALA) to a patient,” issued on Nov. 23, 2021, (7) U.S. Pat. No. 10,603,508 to Boyajian et al., entitled “Adjustable illuminators and methods for photodynamic therapy and diagnosis,” issued on Mar. 31, 2020, and its child, U.S. Patent Application Publication No. 2020/0269063 published Aug. 27, 2020, (8) U.S. Pat. No. 10,357,567 to Lundahl et al., entitled “Methods for photodynamic therapy,” issued on Jul. 23, 2019, and its granted children, U.S. Pat. No. 11,077,192, issued on Aug. 3, 2021 and U.S. Pat. No. 11,135,293 issued on Oct. 5, 2021, and (9) U.S. Patent Application No. 2020/0261580 to Willey entitled “Photodynamic therapy method for skin disorders,” published on Aug. 20, 2020. The entire contents of the foregoing patents and/or patent applications are incorporated herein by reference for background information and the compositions, illuminators, devices, dressings, methods of treatment, processes and techniques relating to photodynamic therapy and diagnosis disclosed therein.

### SUMMARY

The present disclosure describes illuminators for photodynamic therapy and associated techniques and methods of treatment. The illuminators allow improved maneuverability and control for treatment.

According to one embodiment, a method of performing photodynamic therapy is provided. The method includes applying, to the skin of a patient, a topical composition. The topical composition includes 5-aminolevulinic acid (ALA) hydrochloride, and a vehicle comprising at least one chelating agent to enhance accumulation of protoporphyrin IX (PpIX) in the skin. The method further includes incubating the topical composition, and following incubation, applying, to the skin, heat from a heat source for at least a first time period.

In at least one embodiment, the at least one chelating agent is selected from ethylenediaminetetraacetic acid (EDTA) or a pharmaceutically acceptable salt thereof. In at least one embodiment, the method includes, following incubation, exposing the skin to light from a light source for a second time period, wherein the heat is also applied during the second time period. In at least one embodiment, the incubation occurs for between about 2 hours to about 3 hours, and a sum of the first time period and the second time period is about 13 minutes. In at least one embodiment, light is not applied to the skin prior to applying the heat. In at least one embodiment, applying the heat to the skin for 13 minutes increases an amount of PpIX present in the skin by more than 50%. In at least one embodiment, applying the heat to the skin for 13 minutes increases an amount of PpIX present in the skin by more than 80%. In at least one embodiment, the aminolevulinic acid hydrochloride is present in an amount of 20% w/w of the topical composition, and the at least one chelating agent is ethylenediaminetetraacetic acid (EDTA) present in an amount of about 0.1% to about 0.15% of the topical composition.

According to one embodiment, an illuminator is provided with at least one articulated joint which is configured to allow the illuminator panels to be moved approximately 360 degrees in azimuth and then locked into position. In at least one embodiment, two joints are provided to enable 360 degree rotation. According to one embodiment, an illuminator is provided with a control panel or control interface which can be moved relative to the rest of the illuminator. These features allow the illuminator to be positioned in a desired position relative to the patient while allowing a health care provider access to the control panel or interface to administer, control, and monitor treatment.

The panels may be arranged in a variety of configurations to provide illumination to the area of the body being treated. Uniform illumination is desirable in order to impart a uniform therapeutic benefit to the area being treated. Embodiments described below provide a uniformity of greater than approximately 70% at approximately 2 to approximately 4 inches from the treatment surface (such that the measured output over the emitting area is within approximately 70% of the measured maximum over a distance of approximately two to approximately four inches). In at least one embodiment, the uniformity may be between approximately 70% and approximately 80%, e.g., between 72.5% and between 77.5%, over a distance of approximately 2 inches to approximately 4 inches.

In at least one embodiment, the panels may be unfolded and arranged in a flat or substantially flat arrangement to treat areas such as a back or chest and abdomen of a patient. The panels may also be arranged in a U-shape (corresponding to the letter “U”, or substantially similar to the letter “U”) in which the outer panels are parallel or substantially parallel to each other. The panels are configured to be arranged in a U-shaped orientation during treatment, for example, of the head, face, scalp, neck, arms, forearms, hands, feet and/or legs. The panels may be arranged parallel to the floor, perpendicular to the floor, or any other position with respect to the floor, including to treat other portions of a patient (e.g., the torso or back). The panels may have differing orientations such that a first panel may be parallel to the floor and another panel may be oriented at an incline relative to the floor. In at least one embodiment, the panels may be put in a pre-treatment configuration which is a configuration just prior to treatment, e.g., for the purpose of explication and providing a demonstration, instructions or education to the patient about the treatment taking place, and the panels may then be put in a “patient ready” position corresponding to the orientation conducive for treatment.

According to one embodiment, a storage arrangement is provided for an illuminator for photodynamic therapy which allows the illuminator to be folded up into a compact space when not in use. In this embodiment, the illuminator, including arms and panels thereof, when in a stored (stowed) position, does not extend substantially beyond the illuminator base. For example, the illuminator, including arms and panels, when in the stored position, does not extend more than 40%, 30%, 20%, 10%, or 0% beyond the illuminator base. In at least one embodiment, illuminator panels fold around an illuminator pillar in the stored position, thus facilitating a compact storage arrangement. Such a compact storage arrangement is a significant advantage to healthcare providers because the examination and treatment rooms in many medical offices are limited, and the compact storage arrangement provides additional space for patient examination and other types of treatment when the illuminator is not in use. In addition, such a compact arrangement, in conjunction with wheels on the base, allow the illuminator to easily fit through doorways and be moved to other examination and treatment rooms, or other facilities.

The illuminator may be configured to accommodate a patient who is standing, sitting, lying down, or in another position. In at least one embodiment, the panels may be adjusted from a first configuration to a second configuration rapidly. For example, the panels may be adjusted from a first configuration which is conducive to treating a patient's scalp or face to a second configuration which is conducive to treating a patient's back, or vice versa, within a time period of approximately 20-40 seconds or approximately 30 seconds. The various adjustments of the panels and arms can be accomplished quickly (within 30 to 60 seconds) and without tools.

The panels may support, for example, an array of light sources such as light emitting diodes (LEDs). Alternatively, other types of light sources may be used, such as fluorescent or halogen lamps, a non-laser light source, a laser, or other type of light source. The light sources provide illumination which activates a photoactivatable agent as discussed above.

The details of one or more embodiments are set forth in the accompanying drawings and the description below. The figures are provided for the purpose of illustrating one or more embodiments with the explicit understanding that they will not be used to limit the scope or the meaning of the claims. It should be noted that the specific embodiments are not intended as an exhaustive description or as a limitation to the broader aspects discussed herein. One aspect described in conjunction with a particular embodiment is not limited to that embodiment and can be practiced with other embodiment(s).

The following terms are used throughout this patent specification and are as defined below.

As used herein and in the appended claims, singular articles such as “a” and “an” and “the” and similar references in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illustrate the embodiments and does not pose a limitation on the scope of the claims unless otherwise stated. No language in the specification should be construed as indicating any non-claimed element as essential.

The terms “incubation time,” “incubation period,” or “incubation” can refer to the time period from a time when a drug (such as ALA) is applied until a time when a treatment period begins, e.g., when illumination occurs. Speaking generally, an incubation time or incubation period can occur prior to a treatment period. For example, incubation time may be an interval from when a drug is applied (e.g., topically) until the commencement of deliberate exposure to targeted illumination by an illuminator (e.g., as opposed to ambient illumination), or commencement of a treatment step such as applying heat (or applying both heat and light). As will be understood by one of skill in the art, incubation may occur in the dark, which is most common. However, incubation may also occur in the presence of light, including daylight (e.g., so-called painless PDT). Incubation, whether in the dark or under light exposure, may take place with or without heat.

Any embodiment illustratively described herein may suitably be practiced in the absence of any element or elements. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The expression “comprising” means “including, but not limited to.” Thus, other non-mentioned substances, additives, devices or steps may be present.

Unless otherwise indicated, all numbers expressing quantities of properties, parameters, conditions, and so forth, used in the specification and claims are to be understood as being modified in all instances by the terms “approximately” or “about.” Accordingly, unless indicated to the contrary, the numerical parameters set forth in the specification and attached claims are approximations. Any numerical parameter should at least be construed in light of the number of significant digits and by applying ordinary rounding techniques. The terms “approximately” or “about” when used before a numerical designation, e.g., temperature, time, amount, and concentration, including range, indicates approximations which may vary by (+) or (−) 10%, 5% or 1%.

As will be understood by one of skill in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

### BRIEF DESCRIPTION OF THE DRAWINGS

Features, aspects, and advantages of the present invention will become apparent from the following description and the accompanying exemplary embodiments shown in the drawings, which are briefly described below.

FIG. 1 shows a front view of an illuminator system according to an exemplary embodiment.

FIG. 2 shows a top perspective view of the illuminator system of FIG. 1.

FIG. 3 shows a front perspective view of the illuminator system of FIG. 1.

FIG. 4 shows a rear view of the illuminator system of FIG. 1.

FIG. 5 shows a front perspective view of the illuminator system of FIG. 1.

FIG. 6 shows a top perspective view of a mounting mechanism of the illuminator system of FIG. 1.

FIG. 7 shows a side perspective view of a panel of the illuminator system of FIG. 1.

FIG. 8A shows side views of an illuminator of the illuminator system of FIG. 1.

FIG. 8B shows side views of an illuminator of the illuminator system of FIG. 1.

FIG. 9 shows a back view of an illuminator of the illuminator system of FIG. 1.

FIG. 10 shows a back view of an illuminator of the illuminator system of FIG. 1.

FIG. 11 shows a front view of the illuminator system of FIG. 1 in a stored position.

FIG. 12 shows a perspective view of the illuminator system of FIG. 1 in a stored position.

FIG. 13 shows a top view of a panel of the illuminator system of FIG. 1.

FIG. 14 shows a front view of an interface panel of the illuminator system of FIG. 1.

FIG. 15 shows a front view of a main power switch of the illuminator system of FIG. 1.

FIG. 16 shows a front view of a vertical column lock of the illuminator system of FIG. 1.

FIG. 17 shows a front view of an arm lock of the illuminator system of FIG. 1.

FIG. 18 shows a cross-sectional side view of a panel of the illuminator system of FIG. 1.

FIG. 19 shows a perspective view of a fan plenum of a panel of the illuminator system of FIG. 1.

FIG. 20 shows a detailed cross-sectional view of a fan plenum of the illuminator system of FIG. 1.

FIG. 21 shows a detailed cross-sectional view of a fan plenum of the illuminator system of FIG. 1.

FIG. 22 shows a detailed cross-sectional view of a fan plenum of the illuminator system of FIG. 1.

FIG. 23 shows a touch screen of the illuminator system of FIG. 1.

FIG. 24 shows a schematic diagram of a controller of the illuminator system of FIG. 1.

FIG. 25 shows a block diagram of a method of photodynamically diagnosing or treating a patient according to an exemplary embodiment.

FIG. 26 depicts graphical results relating to an amount of accumulated PpiX.

FIG. 27 depicts graphical results relating to an amount of accumulated PpiX.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not limited to that embodiment and can be practiced with any other embodiment(s).

Treatment with Photodynamic Therapy Illuminator

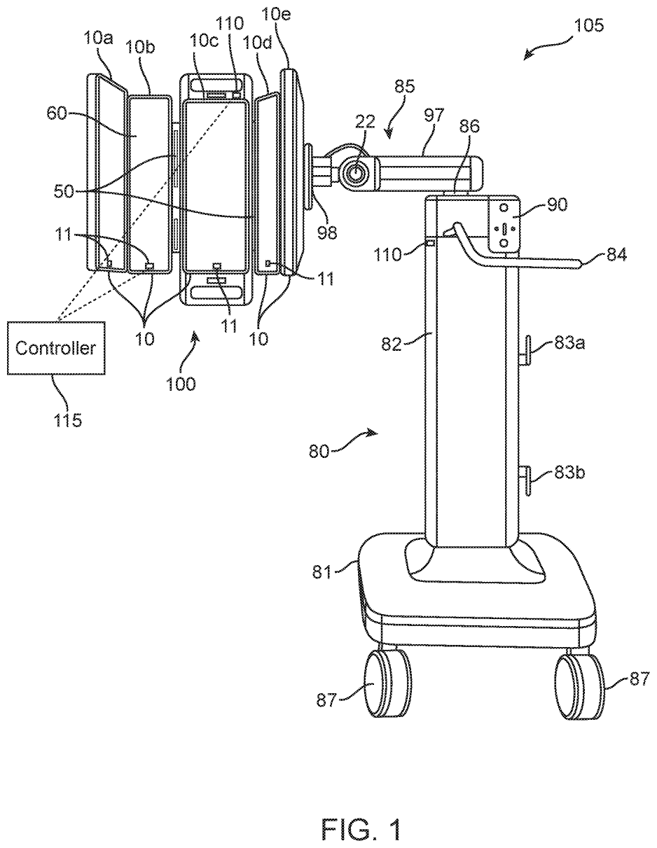

FIGS. 1-4 illustrate at least one embodiment of a configurable illuminator system 105. Illuminator system 105 includes an illuminator 100. The illuminator 100 comprises a plurality of panels 10. The panels 10 are provided with LEDs 60 that are used to emit light for photodynamic therapy for treatment of diseases and disorders of the skin (which may be defined as a multi-layer organ including the epidermis, dermis and subcutaneous tissue, and further including mucous membranes contiguous with the outer skin). The skin to be treated may be, for example, on the surface of the head, face, scalp, neck, arms, legs, torso, genitals, hands or feet, or elsewhere.

In particular, according to at least one embodiment, the head, face or neck may be treated using the illuminator 100. Said treatment of the head, face or neck may occur at a single session all at one time or over multiple sessions over a period of time. The face encompasses, for example, a central portion, eyelids, eyebrows, the periorbital region, the nasal region, lips, chin, mandible, pre-auricular skin, post-auricular skin and sulci. These portions of the face, along with the genitals, hands and feet, may be at a relatively higher risk for developing non-melanoma skin cancer (“NMSC,” which includes all types of skin cancers that are not melanoma, and includes keratinocyte carcinomas). Moderate risk areas include the cheeks, forehead, scalp and neck. For assessing the efficacy of treatment, a lesion count in a given region may be performed in a designated area of the body, such as on the forehead, the left and right cheeks, nose, or chin.

Photodynamic therapy may cause certain individuals to experience discomfort and/or pain. In at least one embodiment, the illuminator provides a gentle flow of air tangential to the skin surface to reduce or minimize pain. The air flow may be provided substantially tangential to (that is substantially parallel to) the skin surface (at an angle of approximately 0 degrees with respect to the skin surface). Thus, a gentle flow is imparted across the surface to be treated. In another embodiment, the air is provided at an angle of 450 or less with respect to the skin surface. In at least one embodiment, the angle may be between approximately 250 and approximately 45°, e.g., approximately 29°, approximately 33°, approximately 37°, or approximately 41°. The air flow may be provided in connection with any treatment method set forth in the present disclosure.

Such an arrangement avoids the air directly impacting the skin surface. Direct impact on the skin surface has been found to cause pain or tingling due to the sensation (e.g., of contact or pressure) against the skin. In at least one embodiment, the gentle flow of air is provided to alleviate pain and/or discomfort that may be experienced by a patient who has undergone PDT with occlusion via a barrier, such as a low density polyethylene (LDPE) or foil barrier. In particular, the flow of cooled air may mitigate pain following administration of ALA to the patient during a treatment cycle.

The following description provides exemplary discussions of how particular areas may be treated using PDT, without limitation. In the following discussion, a distance between a treatment surface and a surface of an illuminator is approximately 2 inches to approximately 4 inches, but it should be appreciated that other distances may be utilized (e.g., between approximately 5 cm to approximately 8 cm). To treat facial lesions, an illuminator as described above may be positioned such that the region to be treated is between approximately 2 to approximately 4 inches from a surface of the illuminator, with the patient's nose not less than approximately 2 inches from the illuminator surface, and the forehead and cheeks no more than approximately 4 inches from the surface. The sides of the patient's face and the patient's ears may be positioned no closer than approximately 2 inches from the illuminator surface, for example.

In at least one embodiment, to treat scalp lesions, an illuminator as described above may be positioned such that the region to be treated is between approximately 2 to approximately 4 inches from a surface of the illuminator, with the patient's scalp not less than approximately 2 inches from the illuminator surface, and no more than approximately 4 inches from the surface. The sides of the patient's face and the patient's ears may be positioned no closer than approximately 2 inches from the illuminator surface, for example.

In at least one embodiment, to treat lesions on the upper extremities, such as the dorsal surface of the hand or the forearms, an illuminator as described above may be positioned such that the region to be treated is between approximately 2 to approximately 4 inches from a surface of the illuminator. Equipment (e.g., a table) may be used to support the upper extremity during light treatment so as to enhance the patient's comfort and stabilize the region to be treated. The aforementioned distances may be employed in connection with carrying out treatment according to any of the embodiments of the present disclosure.

In at least one embodiment, blue light (e.g., light having a wavelength between about 380 nm and about 500 nm) is applied. In at least one embodiment, blue light having a wavelength of about 417 nm (±5 nm) is applied at an intensity of about 10 mW/cm² for 1000 seconds to provide a dose of about 10 J/cm². However, the intensity may be increased (for example, doubled to about 20 mW/cm²) to reduce the treatment time. For example, the intensity may be increased so as to reduce the treatment time by about one-half In other embodiments, red light (such as red light generated by light emitting diodes (LEDs) at, for example, 635 nm) may be used. The red light can provide a dose of, for example, about 10 to about 75 J/cm² (such as 37 J/cm²), e.g., within 10 minutes, or within about 9 to about 11 minutes. The red light may be between about 620 nm and about 750 nm.

In at least one embodiment, the illuminator may irradiate the lesions with a uniform intensity red light for a prescribed period. In certain embodiments, the illuminator irradiates the lesions with a uniform intensity blue light for a first prescribed period and then irradiates the lesions with a uniform intensity red light for a second prescribed period. The wavelength of the irradiating light may be selected to match a wavelength which excites the photoactivatable agent, and preferably has low absorption by non-target tissues. For example, in at least one embodiment, the illuminator is configured to irradiate the lesions with a uniform intensity blue light (e.g., about 417 nm) at a low intensity (e.g., about 0.1 J/cm² to about 2 J/cm²) to photobleach, for example, protoporphyrin IX (PpIX) present at the surface of the patient's skin. In at least one embodiment, the illuminator is configured to irradiate the lesions with a uniform intensity red light (e.g., 635 nm) at a high intensity (e.g., about 30 J/cm² to about 158 J/cm²) to activate PpIX present at deeper layers of the patient's skin, thus avoiding potential damage to the upper layers of the patient's skin.

Furthermore, since the total light dose (J/cm²) is equal to irradiance (mW/cm²) multiplied by time (seconds), an additional parameter to be controlled for delivery of the correct treatment light dose is exposure time (among other parameters which may be controlled to influence treatment). This may be accomplished by a timer, which can control the electrical power supplied to the LED arrays appropriately, and which can be set by a healthcare provider. Data has shown that approximately 10 J/cm² delivered from a source with an irradiance density of 10 mW/cm², or an irradiance density of approximately 9.3 to approximately 10.7 mW/cm², produces clinically acceptable results for desired treatment areas (e.g., the face, scalp, and extremities).

In at least one embodiment, an adjustable illuminator may deliver an irradiance density of approximately 20 mW/cm² for an exposure time of approximately 580 seconds (approximately 8 min., 20 sec) to deliver a clinically acceptable light dose of 10 J/cm². In certain embodiments, a lower intensity may be used with a longer exposure time (e.g., approximately 1,000 seconds of exposure time for a light dose of approximately 10 J/cm²). Alternatively, the adjustable illuminator may include higher power ranges, such as approximately 30 mW/cm², over an exposure time, resulting in a light dose of approximately 10 J/cm². A selected light dose may also be administered by additionally or alternatively varying the irradiance density over treatment time.

### Exemplary Compositions for Photodynamic Therapy

In at least one embodiment, a pharmaceutical composition containing a photoactivatable agent is applied using an applicator, or can be applied by other means, such as glove-protected fingers, a gauze pad, a swab, a bandage, or a spatula. The pharmaceutical composition can be applied in, for example, a topical dosage form (e.g., a compound suitable for administering by applying to a surface of a patient's skin) such as a gel or a solution, and can be applied beyond the lesions to be treated. In at least one embodiment, the photoactivatable agent includes porphyrins or porphyrin precursors.

The amount of the photoactivatable agent in the pharmaceutical composition (e.g., in a dosage form suitable for topical delivery) may vary. In at least one embodiment, the photoactivatable agent is ALA which is present in an amount from about 0.1 wt. % to about 75 wt. %. In at least one embodiment, the amount of the photoactivatable agent in the pharmaceutical composition is greater than about 10 wt. %. In at least one embodiment, the amount of the photoactivatable agent in the pharmaceutical composition is about 20 wt. %. In another embodiment, the amount of the photoactivatable agent in the pharmaceutical composition is greater than zero.

For example, in at least one embodiment, the ALA may be in a liquid solution including about 20% ALA, or in the form of a 10% ALA gel, or a 20% ALA gel. By way of further example, in at least one embodiment, the photoactivatable agent is provided in a gel comprising 20% aminolevulinic acid hydrochloride. In at least one embodiment, the composition in gel form further includes local anesthetics. The pH of the gel formulation may be in a range of about 4.5 to about 7.5.

For example, in at least one embodiment, the composition containing the photoactivatable agent is LEVULAN®, (DUSA Pharmaceuticals, Billerica, MA), a topical formulation of 20% 5-aminolevulinic acid hydrochloride, which may be administered via a KERASTICK® applicator. In at least one embodiment, the composition is AMELUZ® (Biofrontera AG, Leverkusen, Germany), a non-sterile topical formulation of 10% 5-aminolevulinic acid hydrochloride (equaling 7.8% of free acid) in a gel-matrix with nanoemulsion. In at least one embodiment, the photoactivatable agent may be a non-porphyrin agent. In at least one embodiment, about one gram (78 mg) of 5-aminolevulinic acid hydrochloride gel (ALA) is administered.

In at least one embodiment, the composition containing the photoactivatable agent is a composition disclosed in PCT Application No. PCT/IB2022/060058 filed Oct. 19, 2022 and in U.S. patent application Ser. No. 17/968,931 filed Oct. 19, 2022, which are incorporated by reference herein in their entireties for the compositions, compounds and formulae disclosed therein. For example, according to at least one embodiment, the composition containing the photoactivatable agent comprises:
a) a 5-carbon aminoketone compound of Formula I

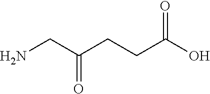

or its pharmaceutically acceptable salt
b) at least one penetration enhancer,
c) at least one chelating agent, and
d) optionally, an antifoaming agent.

Preferably, the at least one penetration enhancer is selected from a group consisting of dialkyl derivatives of acetamide and formamide, pyrrolidone derivatives, fatty acids, fatty acid esters, glycol derivatives, glycerides, azones, polysorbates, macrogolglycerides, polyethylene glycol derivatives, ethoxylated ether derivatives, bile salts and glycosaminoglycan. More preferably, the topical compositions according to the present disclosure comprises dialkyl derivatives of acetamide and formamide such as dimethyl acetamide, dimethyl formamide, pyrrolidone derivatives such as N-methyl-2-Pyrrolidone, fatty acids such as oleic acid, glycol derivatives such as propylene glycol and its fatty esters such as propylene glycol monocaprylate, propylene glycol monolaurate, azones such as laurocapram or 1-n-dodecyl-azacycloheptan-2-one, polysorbates, such as Tween® (polysorbate) 80, macrogolglycerides such as stearoyl macrogolglycerides, oleoyl macrogolglycerides, lauroyi macrogolglycerides, capryl-caproyl macrogolglycerides, polyethylene glycol derivatives such as polyethylene glycol 400, ethoxylated ether derivatives such as diethyleneglycol monoethyl, diethyleneglycol monomethyl ether; and dipropyleneglycol monomethyl ether, glycosaminoglycan such as chondroitin sulfate, keratan sulfate, dermatan sulfide, heparin sulphate and heparan sulphate as the permeation enhancer.

The penetration enhancer is present in the composition in an amount in the range of about 10% w/w to about 50% w/w of the composition, including for example, about 10%, 20%, 30%, 40%, or 50% w/w of the composition and any and all ranges and subranges therein. More preferably, the penetration enhancer is present in the composition in an amount in the range of about 20% w/w to about 40% w/w of the composition, including for example, about 20%, about 30%, or about 40% w/w of the composition and any and all ranges and subranges therein.

In at least one other embodiment, the at least one penetration enhancer is selected from a group consisting of glycol derivatives, polyethylene glycol derivatives, and ethoxylated ether derivatives. In another preferred embodiment, the at least one penetration enhancer is selected from a group consisting of propylene glycol, polyethylene glycol, and 2-(2-Ethoxyethoxy)ethanol (Transcutol®). Propylene glycol is present in the composition in an amount in the range of about 10% w/w to about 50% w/w of the composition, including for example, about 10%, 20%, 30%, 40%, or 50% w/w of the composition and any and all ranges and subranges therein. Preferably, propylene glycol is present in the composition in an amount in the range of about 20% w/w to about 40% w/w of the composition, including for example, about 20%, about 30%, or about 40% w/w of the composition and any and all ranges and subranges therein. 2-(2-Ethoxyethoxy)ethanol (Transcutol®) when used as a penetration enhancer is present in the composition in an amount in the range of about 2% w/w to about 50% w/w of the composition, including for example, about 2%, 4%, 6%, 8%, 10%, 12%, 14%, 16%, 18%, 20%, 22%, 24%, 26%, 28%, 30%, 32%, 34%, 36%, 38%, or 40% w/w of the composition and any and all ranges and subranges therein. Preferably, 2-(2-Ethoxyethoxy)ethanol is present in the composition in an amount in the range of about 4% w/w to about 10% w/w of the composition, including for example, about 4%, 5%, 6%, 7%, 8%, 9% or 10% w/w of the composition and any and all ranges and subranges therein.

In at least one other embodiment, the at least one chelating agent is selected from a group consisting of ethylenediaminetetraacetic acid (EDTA) and its pharmaceutically acceptable salts like disodium edetate, disodium edetate dehydrate, trisodium edetate, di-potassium edetate, dipotassium edetate dehydrate, edetate calcium disodium, diethylenetriamine pentaacetic acid, and organic acid such as citric acid, fumaric acid, malic acid, lactic acid and glycolic acid. Preferably, the at least one chelating agent is disodium edetate.

The at least one chelating agent may be present in the composition in an amount in the range of about 0.01% w/w to about 2% w/w of the composition, including for example, about 0.01%, 0.05%, 0.1%, 0.15%, 0.2%, 0.25%, 0.4%, 0.5%, 0.75%, 0.80%, 0.90%, 1.0%, 1.1%, 1.2%, 1.25%, 1.4%, 1.5%, 1.75%, 1.80%, 1.90% or 2.0% w/w of the composition and any and all ranges and subranges therein. Preferably, the at least one chelating agent is present in an amount in the range of about 0.05% w/w to about 1% w/w, including for example, about 0.05%, 0.1%, 0.15%, 0.2%, 0.25%, 0.4%, 0.5%, 0.75%, 0.80%, 0.90% or 1.0% w/w of the composition and any and all ranges and subranges therein.

For example, EDTA or its pharmaceutically acceptable salt when used as a chelating agent may be present in the composition in an amount in the range of about 0.01% w/w to about 2% w/w of the composition, including for example, about 0.01%, 0.05%, 0.1%, 0.15%, 0.2%, 0.25%, 0.4%, 0.5%, 0.75%, 0.80%, 0.90%, 1.0%, 1.1%, 1.2%, 1.25%, 1.4%, 1.5%, 1.75%, 1.80%, 1.90% or 2.0% w/w of the composition and any and all ranges and subranges therein. Preferably, an amount in the range of about 0.05% w/w to 10% w/w, including for example, about 0.05%, 0.10%, 0.15%, 0.2%, 0.25%, 0.4%, 0.5%, 0.75%, 0.80%, 0.90% or 1.0% w/w of the composition and any and all ranges and subranges therein. In a most preferred embodiment, EDTA or its pharmaceutically acceptable salt is present in the composition in an amount of about 0.1% w/w to about 0.15% w/w of the composition or in an amount of about 0.1% w/w to about 0.25% w/w of the composition.

In at least one embodiment, the 5-carbon aminoketone compound is 5-aminolevulinic acid (ALA) or its pharmaceutically acceptable salt. Preferably, the 5-carbon aminoketone compound is a hydrochloride salt of aminolevulinic acid.

The compound of Formula I

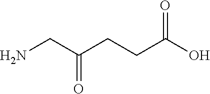

or its pharmaceutically acceptable salt is present in the composition containing the photoactivatable agent in an amount in the range of about 10% w/w to 70% w/w of the composition, including for example, about 10%, 20%, 30%, 40%, 50%, 60% or 70% w/w of the composition. Preferably the compound or its pharmaceutically acceptable salt is present in an amount in the range about 20% w/w to 50% w/w of the composition, including for example, about 20%, 25%, 30%, 35%, 40%, 45% or 50% w/w of the composition. In a most preferred embodiment, the compound of Formula I or its pharmaceutically acceptable salt is present in an amount of about 20% w/w.

The compound of 5-ALA or its pharmaceutically acceptable salt is present in the composition containing the photoactivatable agent in an amount in the range of about 10% w/w to about 70% w/w of the composition, including for example, about 10%, 20%, 30%, 40%, 50%, 60% or 70% w/w of the composition and any and all ranges and subranges therein. Preferably the compound or its pharmaceutically acceptable salt is present in an amount in the range of about 20% w/w to about 50% w/w of the composition including for example, about 20%, 25%, 30%, 35%, 40%, 45% or 50% w/w of the composition and any and all ranges and subranges therein. In a most preferred embodiment, 5-ALA or its pharmaceutically acceptable salt is present in the composition in an amount of about 20% w/w.

In at least one embodiment, the composition containing the photoactivatable agent may contain a variety of other inactive ingredients that are conventionally used in given product types. The inactive ingredients may be selected from alcohol, isopropyl alcohol, polyethylene glycol, propylene glycol, glycerine, diethylene glycol monoethyl ether or purified water or combinations thereof. The composition may further comprise a surfactant or a wetting agent and/or a humectant. The surfactant or wetting agent may be selected from the group consisting of laureth-4, sodium lauryl sulphate, sodium dodecyl sulfate, ammonium lauryl sulphate or sodium octech-1/deceth-1 sulfate thereof. The humectant may be selected from the group consisting of polyethylene glycol, propylene glycol, hyaluronic acid or glycerine thereof.

In at least one embodiment, the composition containing the photoactivatable agent that may be used according to the present disclosure optionally comprise an anti-foaming agent. Suitable anti-foaming agents may include, but are not limited to polydimethylsiloxanes and other silicones, certain alcohols, stearates and glycols. Preferably, the anti-foaming agent is cyclic polydimethylsiloxane. More preferably, the anti-foaming agent is cyclomethicone. The anti-foaming agent is present in the composition in an amount in the range of about 0.2% w/w to about 1.0% w/w of the composition, including for example about 0.2%, 0.25%, 0.4%, 0.5%, 0.75%, 0.80%, 0.90%, or 1.0% w/w of the composition and any and all ranges and subranges therein. Preferably, the anti-foaming agent is present in the composition in an amount in the range of about 0.2% w/w to about 0.5% w/w of the composition. More preferably, the anti-foaming agent is present in the composition in an amount of about 0.5% w/w of the composition.

In at least one embodiment, the composition containing the photoactivatable agent that may be used according to the present disclosure comprises:
a) a 5-carbon aminoketone compound of Formula I

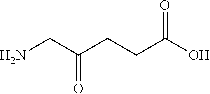

or its pharmaceutically acceptable salt, and
b) a vehicle,
wherein the vehicle comprises:
   (i) at least one penetration enhancer, and
   (ii) at least one chelating agent.

In at least one embodiment, the vehicle comprises an optional antifoaming agent.

In another embodiment, the composition containing the photoactivatable agent that may be used according to the present disclosure comprises:
a) a 5-carbon aminoketone compound of Formula I

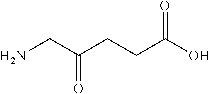

or its pharmaceutically acceptable salt in the form of a dry solid, and
b) a vehicle,
wherein the vehicle comprises:
   (i) at least one penetration enhancer,
   (ii) at least one chelating agent, and
   (iii) optionally, an antifoaming agent.

In at least one embodiment, the composition containing the photoactivatable agent that may be used according to the present disclosure comprises:
a) a 5-carbon aminoketone compound of Formula I

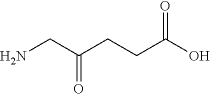

or its pharmaceutically acceptable salt in the form of a dry solid, and
b) a vehicle,
wherein the vehicle comprises:
   (i) at least one penetration enhancer selected from a group consisting of glycol derivatives, polyethylene glycol derivatives, ethoxylated ether derivatives,
   (ii) ethylenediaminetetraacetic acid (EDTA) or its pharmaceutically acceptable salts thereof, and
   (iii) optionally, an antifoaming agent.

In at least one other embodiment, the composition containing the photoactivatable agent that may be used according to the present disclosure comprises:
a) 5-ALA or its pharmaceutically acceptable salt,
b) propylene glycol,
c) EDTA or its pharmaceutically acceptable salt, and
d) optionally, an antifoaming agent.

In yet at least one other embodiment, the composition containing the photoactivatable agent that may be used according to the present disclosure comprises:
a) 5-ALA or its pharmaceutically acceptable salt, in the form of a dry solid, and
b) a vehicle,
wherein the vehicle comprises:
   (i) propylene glycol,
   (ii) EDTA and pharmaceutically acceptable salts, and
   (iii) optionally, an antifoaming agent.

In yet another embodiment, the composition containing the photoactivatable agent that may be used according to the present disclosure comprises:
a) a 5-carbon aminoketone compound of Formula I

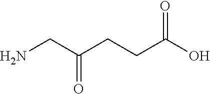

or its pharmaceutically acceptable salt in the form of a dry solid, and
b) a vehicle,
wherein the vehicle comprises:
   (i) propylene glycol,
   (ii) 2-(2-Ethoxyethoxy)ethanol, and
   (iii) disodium edetate, and
   (iv) optionally, an antifoaming agent.

In at least one embodiment, the composition containing the photoactivatable agent that may be used according to the present disclosure comprises:
a) 5-ALA or its pharmaceutically acceptable salt in the form of a dry solid, and
b) a vehicle,
wherein the vehicle comprises:
   (i) propylene glycol,
   (ii) 2-(2-Ethoxyethoxy)ethanol, and
   (iii) disodium edetate, and
   (iv) optionally, an antifoaming agent.

In at least one embodiment, the composition containing the photoactivatable agent that may be used according to the present disclosure comprises:
a) a 5-carbon aminoketone compound of Formula I

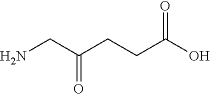

or its pharmaceutically acceptable salt in the form of a dry solid, and
b) a vehicle,
wherein the vehicle comprises:
   (i) propylene glycol in an amount in the range of about 10% w/w to about 50% w/w,
   (ii) 2-(2-Ethoxyethoxy)ethanol in an amount in the range of about 2% w/w to about 50% w/w, and
   (iii) disodium edetate, and
   (iv) optionally, an antifoaming agent.

In at least one other embodiment, the composition containing the photoactivatable agent that may be used according to the present disclosure comprises:
a) 5-ALA or its pharmaceutically acceptable salt in the form of a dry solid, and
b) a vehicle,
wherein the vehicle comprises:
   (i) propylene glycol in an amount in the range of about 10% w/w to about 50% w/w,
   (ii) 2-(2-Ethoxyethoxy)ethanol in an amount in the range of about 2% w/w to about 50% w/w, and
   (iii) disodium edetate, and
   (iv) optionally, an antifoaming agent.

In at least one embodiment, the composition containing the photoactivatable agent that may be used according to the present disclosure comprises:
a) a 5-carbon aminoketone compound of Formula I or its pharmaceutically acceptable salt,

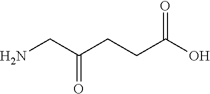

b) propylene glycol,
c) 2-(2-Ethoxyethoxy)ethanol, and
d) disodium edetate.

In at least one embodiment, the composition containing the photoactivatable agent that may be used according to the present disclosure comprises:
a) 5-aminolevulinic acid in an amount of 20% w/w of the composition,
b) propylene glycol in an amount of 20% to 40% w/w of the composition, and
c) EDTA in an amount of 0.1% to 0.5% w/w of the composition.

In at least one embodiment, the composition containing the photoactivatable agent further comprises 2-(2-Ethoxyethoxy) ethanol in an amount of 4% to 10% w/w of the composition.

In at least one other embodiment, the composition containing the photoactivatable agent further comprises cyclomethicone in an amount of 0.2 to 0.5% w/w of the composition.

In at least one embodiment, the composition containing the photoactivatable agent that may be used according to the present disclosure comprises:
a) a 5-carbon aminoketone compound of Formula I

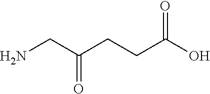

or its pharmaceutically acceptable salt, in an amount of 1-30% w/w,
b) propylene glycol in an amount in the range of about 10% w/w to about 50% w/w,
c) 2-(2-Ethoxyethoxy)ethanol in an amount in the range of about 2% w/w to about 50% w/w, and
d) disodium edetate.

In at least one embodiment, the composition containing the photoactivatable agent that may be used according to the present disclosure comprises:
a) a 5-carbon aminoketone compound of Formula I

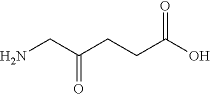

or its pharmaceutically acceptable salt,
b) ethanol,
c) Laureth-4
d) polyethylene glycol,
e) isopropyl alcohol,
f) propylene glycol,
g) 2-(2-Ethoxyethoxy)ethanol,
h) edetate disodium,
i) cyclomethicone, and
j) purified water.

In at least one embodiment, the composition containing the photoactivatable agent that may be used according to the present disclosure comprises:
a) a 5-carbon aminoketone compound of Formula I

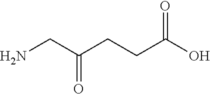

or its pharmaceutically acceptable salt in the form of a dry solid, and
b) a vehicle,
wherein the vehicle comprises:
   (i) ethanol,
   (ii) Laureth-4,
   (iii) polyethylene glycol,
   (iv) isopropyl alcohol,
   (v) propylene glycol,
   (vi) 2-(2-Ethoxyethoxy)ethanol,
   (vii) edetate disodium,
   (viii) cyclomethicone, and
   (ix) purified water.

In at least one other embodiment, the composition containing the photoactivatable agent that may be used according to the present disclosure comprises:
a) 5-ALA or its pharmaceutically acceptable salt,
b) ethanol,
c) Laureth-4,
d) polyethylene glycol,
e) isopropyl alcohol,
f) propylene glycol,
g) 2-(2-Ethoxyethoxy)ethanol,
h) edetate disodium,
i) cyclomethicone, and
j) purified water.

In at least one embodiment, the composition containing the photoactivatable agent that may be used according to the present disclosure comprises:
a) 5-ALA or its pharmaceutically acceptable salt in the form of a dry solid, and
b) a vehicle,
wherein the vehicle comprises:
   (i) ethanol,
   (ii) Laureth-4,
   (iii) polyethylene glycol,
   (iv) isopropyl alcohol,
   (v) propylene glycol,
   (vi) 2-(2-Ethoxyethoxy)ethanol,
   (vii) edetate disodium,
   (viii) cyclomethicone, and
   (ix) purified water.

In at least one embodiment, the composition containing the photoactivatable agent that may be used according to the present disclosure comprises:
a) a 5-carbon aminoketone compound of Formula I or its pharmaceutically acceptable salt in an amount of 20% w/w,

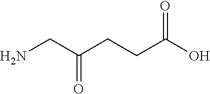

b) ethanol in an amount of 10 to 15% w/w of the composition,
c) Laureth-4 in an amount of 5 to 10% w/w of the composition,
d) polyethylene glycol in an amount of 1 to 5% w/w of the composition,
e) isopropyl alcohol in an amount of 2 to 4% w/w of the composition,
f) propylene glycol in an amount of 20 to 40% w/w of the composition,
g) 2-(2-Ethoxyethoxy)ethanol in an amount of 2 to 4% w/w of the composition,
h) edetate disodium in an amount of 0.1 to 0.25% w/w of the composition,
i) cyclomethicone in an amount of 0.2 to 0.5% w/w of the composition, and
j) purified water.

In at least one other preferred embodiment, the composition containing the photoactivatable agent that may be used according to the present disclosure comprises:
a) 5-ALA or its pharmaceutically acceptable salt in an amount of 20% w/w,
b) ethanol in an amount of 10 to 15% w/w of the composition,
c) Laureth-4 in an amount of 5 to 10% w/w of the composition,
d) polyethylene glycol in an amount of 1 to 5% w/w of the composition,
e) isopropyl alcohol in an amount of 2 to 4% w/w of the composition,
f) propylene glycol in an amount of 20 to 40% w/w of the composition,
g) 2-(2-Ethoxyethoxy)ethanol in an amount of 2 to 4% w/w of the composition,
h) edetate disodium in an amount of 0.1 to 0.25% w/w of the composition,
i) cyclomethicone in an amount of 0.2 to 0.5% w/w of the composition, and
j) purified water.

In at least one embodiment, the composition containing the photoactivatable agent that may be used according to the present disclosure comprises:
a) a 5-carbon aminoketone compound of Formula I

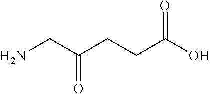

or its pharmaceutically acceptable salt in the form of a dry solid, and
b) a vehicle,
wherein the vehicle comprises:
   (i) ethanol in an amount of 10 to 15% w/w of the composition,
   (ii) Laureth-4 in an amount of 5 to 10% w/w of the composition,
   (iii) polyethylene glycol in an amount of 1 to 5% w/w of the composition,
   (iv) isopropyl alcohol in an amount of 2 to 4% w/w of the composition,
   (v) propylene glycol in an amount of 20 to 40% w/w of the composition,
   (vi) 2-(2-Ethoxyethoxy)ethanol in an amount of 2 to 4% w/w of the composition,
   (vii) edetate disodium in an amount of 0.1 to 0.25% w/w of the composition, and
   (viii) cyclomethicone in an amount of 0.2 to 0.5% w/w of the composition.

### Exemplary Treatment Methods

In at least one embodiment, heating of the skin is performed to enhance the efficacy of photodynamic therapy. Enhanced efficacy is correlated with a reduction in incubation time for a photoactivatable agent and increased absorption of the photoactivatable agent. In addition, the enhanced efficacy is reflected by an increase in complete incision rates when used for skin cancer. Generally speaking, in at least one embodiment, such results are realized by administering heat to an affected area; administering a therapeutically effective dose of a pharmaceutical composition; and administering a light dose to the affected area to treat a disease or disorder or of the skin. Treatment may be further enhanced through pain alleviation.

In at least one embodiment, the time between application of heat to the affected area and application of the ALA may vary. This is known as the “heat-to-drug interval” and may be seconds, minutes, hours or even days. In at least one embodiment, the heat-to-drug interval is about 1 second to about 60 seconds. In at least one embodiment, the heat-to-drug interval is about 1 hour to about 24 hours. In at least one embodiment, the “drug-to-light” interval reflects the period between administration of the photoactivatable agent and the administration of light (e.g., from illuminator 100). The “duration of exposure” or “exposure time” is the amount of time the skin is continuously exposed (e.g., to a pharmaceutical composition, to illumination, etc.).

The ALA can be applied to the surface to be treated (e.g., directly to lesions to be treated) and to a margin beyond the lesions (such as approximately 5 mm or less than approximately 5 mm, e.g., approximately 2-4 mm). The ALA can be administered to affected areas, without applying the ALA to healthy tissue not containing lesions and/or areas away from the lesions. In certain applications, the ALA may be covered with a barrier, such as a low density polyethylene or foil barrier. The barrier may be provided in a kit with an adhesive, a netting or a mesh to help secure the barrier in place. In at least one embodiment, the ALA may be covered, following its application to the treatment surface, by a material having a degree of occlusion of 65% or more, a material having a degree of occlusion of 75% or more, or 85% or more, or another material. Such material may be provided in order to retain moisture in the tissue and thus improve penetration of the ALA. The low density polyethylene may be characterized by a density of approximately 0.917 g/cm³ to approximately 0.930 g/cm³.

As discussed further below, in at least one embodiment, treatment may be carried out on heat-treated skin. To heat the skin, a heating element (e.g., a heat source) may be provided that is separate from or integrated with illuminator 100. The heat source may be used to heat the region to be treated. According to one embodiment, a method of treatment includes warming up an illuminator so as to cause heat to be emitted from the illuminator, and exposing a treatment site to the illuminator. Heating is believed to increase the rate of porphyrin production in the skin. In particular, the heat accelerates the conversion of ALA to porphyrin (e.g., photoactivatable porphyrin or proto porphyrin). The relationship between temperature exposure and ALA conversion is non-linear, and the enzymatic pathways responsible for the conversion are highly sensitive to temperature.

In at least one embodiment, increasing the temperature by approximately 2° C. may approximately double the rate of production of protoporphyrin IX (PpIX), for example. In at least one embodiment, by heating the skin as described herein, the rate of porphyrin production in the skin is increased by about 10%, about 20%, about 30%, about 40%, about 50% or more, about 60% or more, about 70% or more, about 80% or more, or about 85% or more. In at least one embodiment, the rate of porphyrin production in the skin is increased by about 50% or more, about 60% or more, about 70% or more, about 80% or more, or about 85% or more by heating the skin for five minutes followed by heating and a light dose for about eight minutes or about 8 minutes and 20 seconds. For example, as described in more detail below, the amount of PpIX in skin can approximately double with the application of heat to the treatment area compared to when no heat is applied to the treatment area.

In at least one embodiment, the heat may be applied before or during illumination with the illuminator 100. For example, first, the ALA may be applied. Next, the heating element may be activated, to apply heat to the patient's skin for a first treatment period for a thermal soak, which may be approximately 20 to approximately 30 minutes, for example, or another interval. During the heating, the treatment site may or may not be occluded. The treatment site may be heated while being occluded. In at least one embodiment, the thermal soak may be between approximately 18 minutes and approximately 32 minutes and may or may not be commensurate with the first treatment period. Following the first treatment period, light may be applied for a second treatment period, e.g., about 8 minutes to about 15 minutes. In at least one embodiment, light may be applied for 8 minutes and 20 seconds. The total thermal soak, corresponding to exposure to heat, may be between about 1 minute and about 90 minutes.

In at least one embodiment, the skin is heated to a surface temperature of greater than about 37° C. In at least one embodiment, the skin is heated to a surface temperature of greater than about 40° C.

The present disclosure thus provides a method for photodynamically treating a surface of a patient (and optionally occluding the patient's skin as part of treatment). The patient may be illuminated to treat actinic keratosis (AKs), disseminated superficial actinic porokeratosis (DSAP) or refractory disseminated porokeratosis, acne (e.g., cystic acne, inflammatory acne, non-inflammatory acne), photo-damaged skin, skin cancer (e.g., non-melanoma skin cancer (NMSC), nodular basal cell carcinoma, recurrent nodular basal cell carcinoma, infiltrative basal cell carcinoma, multi-focal basal cell carcinoma), warts, psoriasis, or other dermatological conditions.

For example, in at least one embodiment, the LEDs of illuminator 100 emit light for photodynamic treatment of cystic acne. In particular, the LEDs may emit red light for carrying out PDT of acne, NMSC, AK, or DSAP on heat-treated skin (e.g., skin that is previously or concurrently heated). In at least one embodiment, cystic acne may be treated by applying 10% ALA gel and delivering a light dose of 37 J/cm² of light at 630 nm for approximately one hour, while heating or otherwise maintaining a surface of the patient to be treated (a treatment surface, skin surface, etc.) at a temperature of approximately 40° C.

In at least one embodiment, by heating the skin as described herein, a reduction in incubation time needed for ALA may be achieved. Traditional PDT for AK requires a fourteen (14) hour incubation with ALA before exposure to blue light. In at least one embodiment, the incubation period may be drastically reduced. For example, the incubation period may be reduced to less than about 30 minutes, about 30 minutes, about 45 minutes or about 1 hour. A significant quantity of porphyrins is produced after 20 minutes of incubation of 20% ALA gel on skin heated to about 40° C., with even a greater quantity produced after about 30 minutes. The quantity of porphyrins produced after 60 minutes incubation of 20% ALA gel without heat is smaller than for either 20 or 30 minutes with heat.

Thus, the reaction to photodynamic therapy is significantly greater for heated skin of a patient than for unheated skin. In at least one embodiment, the heat source may be used for heating for a period of between about 15 to about 60 minutes. In at least one embodiment, the incubation period (e.g., an incubation time, as discussed further herein) may be about 17 minutes for 20% ALA gel with a heat source achieving a skin temperature of between about 38° C. and about 42° C. In at least one embodiment, a one hour incubation period may be employed for 10% ALA, where the heat source is a sodium acetate warming mask used to heat the skin to about 40° C., followed by a light dose of 37 J/cm² light at 635 nm (e.g., for treating moderate inflammatory or pustular acne). A reduction in lesion counts was observed nine months following even a single photodynamic therapy treatment session. In at least one embodiment, a one hour incubation period of 20% ALA is performed, where the heat source is a heating pad or a sodium acetate warming pouch.

A method of treating skin diseases or disorders using photodynamic therapy (e.g., with red light) on pre-heated skin may be carried out using an illuminator according to the present disclosure. In at least one embodiment, the pharmaceutical composition is a nanoemulsion comprising 10% 5-aminolevulinic acid HCl. In at least one embodiment, the light has a wavelength of between about 620 to about 640 nm, and more particularly, about 630 nm. In one embodiment, the suitable dose of light is about 37 J/cm².

For example, a method of treating facial acne in a subject in need thereof may be carried out, including (i) applying heat to an affected area of the subject's skin using a heat source to achieve a skin temperature of between about 38° C. and about 42° C. for a suitable time; (ii) incubating a pharmaceutical composition comprising a photoactive agent for a period of less than about 14 hours; (iii) applying a therapeutically effective amount of the incubated pharmaceutical composition to the affected area; and (iv) administering light (e.g., red light) to the affected area to treat the facial acne. In at least one embodiment, the acne is mild acne, moderate acne or severe acne. In at least one embodiment, the heat source is a heat mask such as an acetate mask that heats upon crystallization. In at least one embodiment, the affected area is heated for about 60 minutes.

In at least one embodiment, the affected area is heated to about 40° C. In at least one embodiment, the incubation period is less than about 3 hours but achieves equivalent efficacy as if the pharmaceutical composition had been incubated for about 3 hours in the absence of applying heat (i.e., without heating) to achieve a skin temperature of between about 38° C. and about 42° C. In at least one embodiment, the incubation period is less than about 1 hour but achieves equivalent efficacy as if the pharmaceutical composition had been incubated for about 1 hour in the absence of heating to achieve a skin temperature of between about 38° C. and about 42° C. In an exemplary embodiment, the incubation period is less than about 15 minutes, less than about 10 minutes, or less than about 5 minutes.

In at least one embodiment, the treatment results in a reduction in acne lesion count for a patient suffering from acne. In at least one embodiment, the reduction persists for a period of at least three months. In exemplary embodiments, treatment results in a reduction in acne lesion severity. In at least one embodiment, the reduction persists for a period of at least three months. In at least one embodiment, the side effects of treatment are reduced relative to a method that does not include heating of the skin.

In at least one embodiment, a method is provided for treating non-melanoma skin cancers (NMSCs) of the face. The method includes applying heat to achieve a skin temperature of between about 38° C. and about 42° C.; incubating a pharmaceutical composition for less than about 14 hours; applying a therapeutically effective amount of the composition to the affected area, and administering a suitable dose of light (e.g., red light) to the area. In at least one embodiment, the non-melanoma skin cancer is basal cell carcinoma. In at least one embodiment, the non-melanoma skin cancer is a squamous cell carcinoma (SCC). In at least one embodiment, the non-melanoma skin cancer is a basal cell carcinoma, and heat is applied for about thirty minutes or for about twenty minutes. In at least one embodiment, the incubation period is (i) less than about 14 hours but achieves equivalent efficacy as if the pharmaceutical composition had been incubated for about 14 hours in the absence of heating; (ii) less than about 3 hours but achieves equivalent efficacy as if the pharmaceutical composition had been incubated for about 3 hours in the absence of heating; or (iii) less than about ten minutes.

In at least one embodiment, a method of treating AKs of the face is provided. The method includes applying heat to achieve a skin temperature of between about 38° C. and about 42° C.; incubating a pharmaceutical composition for less than about 14 hours; applying a therapeutically effective amount of the composition to the affected area, and administering a suitable dose of light (e.g., red light) to the area. In at least one embodiment, the affected area is heated for about 30 minutes. In at least one embodiment, the affected area is heated to about 40° C. In at least one embodiment, the incubation period is (i) less than about 14 hours but achieves equivalent efficacy as if the pharmaceutical composition had been incubated for about 14 hours in the absence of heating, (ii) less than about 3 hours but achieves equivalent efficacy as if the pharmaceutical composition had been incubated for about 3 hours in the absence of heating, (iii) less than about 1 hour but achieves equivalent efficacy as if the pharmaceutical composition had been incubated for about 1 hour in the absence of heating, or (iv) less than about 10 minutes.

In at least one embodiment, a method of treating disseminated superficial actinic porokeratosis (DSAP) of the face in a subject in need thereof is provided. The method includes applying heat to achieve a skin temperature of between about 38° C. and about 42° C.; incubating a pharmaceutical composition for less than about 14 hours; applying a therapeutically effective amount of the composition to the affected area, and administering a suitable dose of light (e.g., red light) to the area. In at least one embodiment, the incubation period is (i) less than about 14 hours but achieves equivalent efficacy as if the pharmaceutical composition had been incubated for about 14 hours in the absence of heat, (ii) less than about 3 hours but achieves equivalent efficacy as if the pharmaceutical composition had been incubated for about 3 hours in the absence of heat, (iii) less than about 1 hour but achieves equivalent efficacy as if the pharmaceutical composition had been incubated for about 1 hour in the absence of heat, or (iv) less than about 10 minutes.

In at least one embodiment, the ALA is incubated simultaneously with application of heat to the skin or within a few seconds or minutes prior to heating of the skin, or after heating of the skin has commenced. The following discussion sets forth illustrative examples of how treating a disease or disorder of the skin may be carried out using an exemplary illuminator, e.g., for performing photodynamic therapy, in combination with heat.

### Examples—Effect of Heat on PpIX Levels in Skin

A study was conducted to evaluate the effect of heat application on PpIX levels in skin after a photoactivatable agent is applied to the skin. More specifically, the study was performed on porcine test subjects to investigate the pharmacokinetic profiles of 5-ALA and PpIX for enhanced topical formulations using Levulan® (i.e., Levulan® as enhanced, for example, with a chelating agent as discussed herein). Three compositions were tested, including (i) Levulan® (DUSA Pharmaceuticals, Inc., Billerica, MA), (ii) Levulan® with 0.1% ethylenediaminetetraacetic acid (EDTA) present in an amount of about 0.1% w/w of the topical composition and (iii) Levulan® with EDTA present in an amount of about 0.15% w/w of the topical composition.

Each formulation was prepared for application via the Levulan® Kerastick® applicator. The same batch of each formulation (e.g., same batch number, manufacturing date, and expiration date) was used for each respective porcine test subject. The formulations were stored at temperatures between 20° C. to 25° C. (680-77° F.). Testing was performed on thirty-six (36) test subjects (Sus scrofa domesticus), allowing for three test subjects per time point (two hours or three hours) per formulation, with heat and without heat. PpIX was evaluated after each time point (i.e., after two hours and after three hours of incubation).

The test subjects were kept in an environment having a temperature between 18° C. to 28° C. with a humidity between 30%-70%. In the day when treatment was performed, the test subjects were exposed to approximately 12 hours of darkness and approximately 12 hours of light. The test subjects were protected from light during the test incubation period (of either two or three hours).

The test subjects were divided into 12 groups as shown Table 1:

TABLE 1
Study groups

| | |
|---|---|
| Group 1 | 3 test subjects: Levulan ®; no heat; 2 hrs |
| Group 2 | 3 test subjects: Levulan ®; no heat; 3 hrs |
| Group 3 | 3 test subjects: Enhanced 0.1% EDTA; no heat; |
| | 2 hrs |
| Group 4 | 3 test subjects: Enhanced 0.1% EDTA; no heat; |
| | 3 hrs |
| Group 5 | 3 test subjects: Enhanced 0.15% EDTA; no heat; |
| | 2 hrs |
| Group 6 | 3 test subjects: Enhanced 0.15% EDTA; no heat; |
| | 3 hrs |
| Group 7 | 3 test subjects: Levulan ®; with heat; 2 hrs |
| Group 8 | 3 test subjects: Levulan ®; with heat; 3 hrs |
| Group 9 | 3 test subjects: Enhanced 0.1% EDTA; with heat; |
| | 2 hrs |
| Group 10 | 3 test subjects: Enhanced 0.1% EDTA; with heat; |
| | 3 hrs |
| Group 11 | 3 test subjects: Enhanced 0.15% EDTA; with heat; |
| | 2 hrs |
| Group 12 | 3 test subjects: Enhanced 0.15% EDTA; with heat; |
| | 3 hrs |

A portion of the dorso-lateral trunk skin of each test subject area was divided into ten blocks for dose application, where each block was approximately 2 cm×2 cm, with four cm of space between blocks. The test sites were cleaned with ethanol prior to dose application.

Each dose (of the three formulations) was applied topically per Levulan® dosing instructions to different blocks. A single dose included two applications using the Kerastick® applicator of approximately 15 seconds each, with an interval of approximately two minutes between applications. The duration of treatment was confined to a single dose.

Following application of the dose, heat was applied to the skin of test subjects in groups 7-12. Heat was applied at approximately 39° C. to approximately 41° C. for approximately thirteen minutes after the respective group of test subjects were dosed. More particularly, test subjects of groups 7-12 were subjected to heat application for about 13 minutes after formulation application and the designated incubation time. Infrared (IR) lamps were used to provide the heat.

After 2 hours of incubation, dermis and epidermis samples were taken from the test subjects designated for the 2 hour time point (e.g., groups 1, 3, 5, 7, 9, and 11) and tested for amounts of PpIX. After 3 hours of incubation, dermis and epidermis samples were taken from the test subjects designated for the 3 hour time point (e.g., groups 2, 4, 6, 8, 10, and 12) and tested for amounts of PpIX.

The epidermis and dermis layers were separated approximately 10-20 minutes after harvesting the stratum corneum of the subjects. To separate the epidermis and the dermis, the skin samples were then kept in a hot air oven for 5-10 minutes at 60° C.-62° C. in a closed aluminum foil. The epidermis layer was separated from the dermis layer manually. The dermis and epidermis layers were collected in separate centrifuge containers. The container was weighed before and after adding the skin layer. The difference in weight was calculated to determine the weight of the skin layer. Skin samples were flash frozen immediately after skin layer separation using liquid nitrogen to stop continued production of PpIX in the samples. The samples remained frozen for at least 24 hours prior to processing for analysis. The aforementioned processes were carried out under monochromatic light (i.e., a sodium vapor lamp). A homogenization solution was added to the containers containing the skin tissues to prepare 4% w/v tissue homogenate, and the skin tissue homogenate was prepared using homogenizer under constant cooling using an ice bath. After every run, a probe of the homogenizer was washed and dried. The tissue homogenate samples were analyzed for 5-ALA and PpIX.

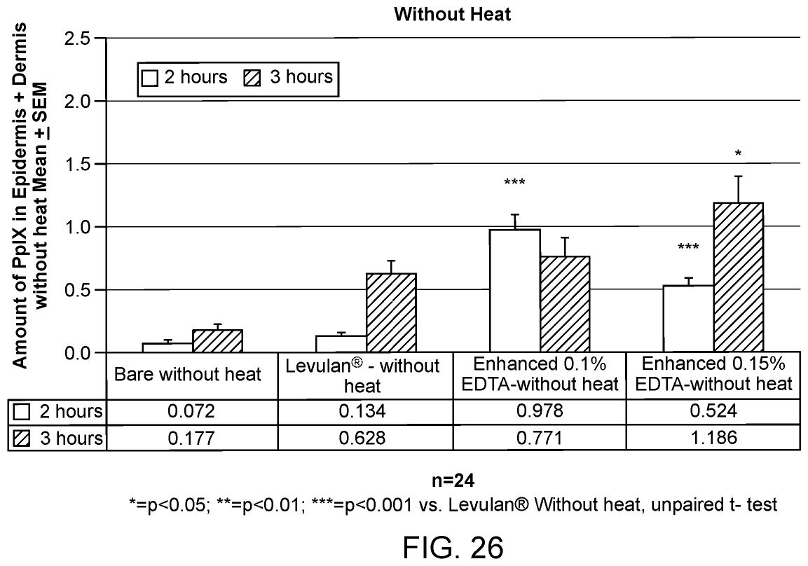

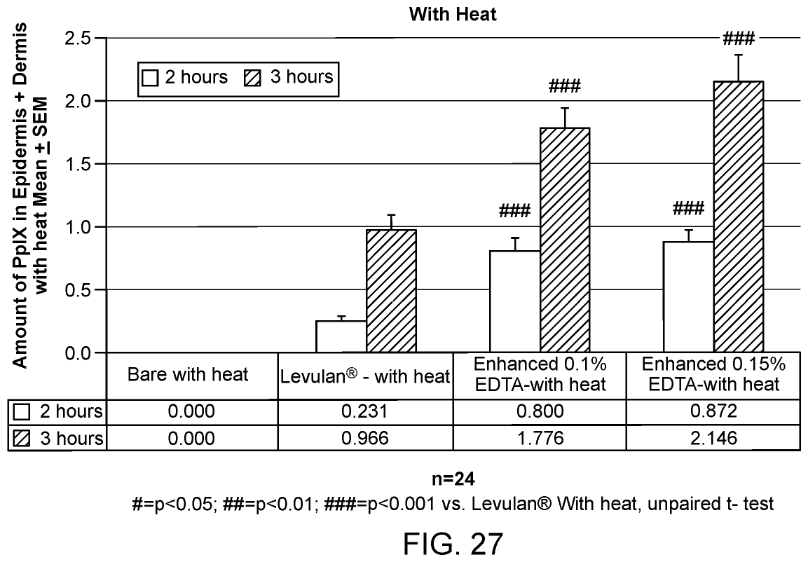

As shown in FIGS. 26-27, exposing the formulations to heat resulted in higher PpIX levels at 3 hours of incubation. For example, application of the reference Levulan® with no heat applied produced about 0.628 mcg/g of PpIX after 3 hours of incubation. Application of Levulan® with approximately 13 minutes of heat applied produced about 0.966 mcg/g of PpIX after 3 hours of incubation. As such, the treatment with the heat resulted in over a 50% increase in the amount of PpIX produced that the treatment without heat.

For another example, application of the Levulan® enhanced with 0.10% EDTA with no heat applied produced about 0.771 mcg/g of PpIX after 3 hours of incubation. Application of the Levulan® enhanced with 0.1% EDTA with 13 minutes of heat applied produced about 1.776 mcg/g of PpIX after 3 hours of incubation. As such, the treatment with the heat resulted in over double the amount of PpIX than the treatment without heat.

Application of the Levulan® enhanced with 0.15% EDTA with no heat applied produced about 1.186 mcg/g of PpIX after 3 hours of incubation. Application of the Levulan® enhanced with 0.15% EDTA with 13 minutes of heat applied produced about 2.146 mcg/g of PpIX after 3 hours of incubation. As such, the treatment with the heat resulted in over an 80% increase in the amount of PpIX produced than the treatment without heat.

This surprising increase in PpIX with such a short duration of heating has not been previously achieved and was not expected previously. The short duration of heating according to the techniques of the present disclosure overcomes a major drawback of PDT generally, namely, the necessity for a patient to be exposed to an illuminator for a long period of time.

The foregoing examples are believed to allow for dramatic reductions in total treatment time to be realized, with concomitant reduction in pain.

In particular, the compositions described above may be utilized to treat patients in accelerated PDT protocols. For example, in at least one embodiment, a method of performing PDT (e.g., for treatment of a dermatological disorder) can be carried out in which topical composition is applied to skin and incubated for a predetermined incubation period.

In at least one embodiment, a topical composition as enhanced with the at least one chelating agent is applied to the skin, e.g., with an applicator. The topical composition may comprise ALA (e.g., ALA HCl) as enhanced with at least one chelating agent in accordance with any of the embodiments of the present disclosure, such as EDTA in an amount of about 0.1% w/w to about 0.15% w/w of the composition.

Following topical application, the incubation period may be about 30 minutes to about 3 hours, which in some embodiments, may be between about 2 hours to about 3 hours. Optionally, the skin may be occluded during all or part of the incubation. In some embodiments, the skin is occluded with a barrier, such as a low density polyethylene (LDPE) barrier or foil barrier, following application of ALA as enhanced with at least one chelating agent.

After a predetermined incubation period, the skin is exposed to heat from a heat source, and, in some embodiments, light (e.g., from a light source). For example, heat may be applied during an initial part of treatment, followed by both light and heat being applied during a final part of treatment. Optionally, a flow of air may be directed to the skin during the initial and/or final parts of treatment which may alleviate associated pain. For example, a gentle flow of air may be provided during at least the initial part of treatment (e.g., a first time period).

In some embodiments, the patient may be exposed to about 5 minutes of heat, followed by about 8 minutes of both light and heat. In some embodiments, 8 minutes and 20 seconds of light and heat may be administered. Thus, a sum of the initial part of treatment and the final part of treatment may be about 13 minutes. The light can be, e.g., blue light applied at an intensity of 20 mW/cm² or about 30 mW/cm². In some embodiments, blue light may be applied at an intensity of 10 mW/cm². The blue light may be supplied for a sufficient time period to provide a dose of about 10 J/cm². In some embodiments, red light may be applied to achieve a dose of about 10 J/cm² to about 75 J/cm², e.g., within less than 10 minutes. In some embodiments, the patient may be exposed to both blue and red light. Any combination of the light dosages, durations and/or intensities set forth in the present disclosure may be utilized.

In some embodiments, a patient may be exposed to heat for a duration of about 4, about 5, about 6, about 7, about 8, about 9, about 10, about 11, about 12, about 13, about 14, about 15, about 16, about 17, about 18, about 19 or about 20 minutes in the initial part of the treatment. Following the initial treatment, the patient may be exposed to both heat and light for the final part of the treatment, which may be for a duration of about 4, about 5, about 6, about 7, about 8, about 9, about 10, about 11, about 12, about 13, about 14, about 15, about 16, about 17, about 18, about 19 or about 20 minutes. In some embodiments, the total heating time may be from between about 10 minutes to about 30 minutes.

In some embodiments, the illumination can be performed with an illuminator according to the disclosed embodiments described herein. In some embodiments, a kit is provided including the illuminator, the heat source and at least one applicator having a topical composition according to the foregoing embodiments.

### Illuminator Panel Configuration

Returning to FIGS. 1-4, in at least one embodiment, the illuminator 100 preferably has five panels 10 (namely, panels 10a-10e). The panels 10 may vary in size. For example, a first panel 10a may be a first size, a second panel 10b may be a second size, a third panel 10c may be a third size, a fourth panel 10d may be the second size, and a fifth panel 10e may be the first size. In at least one alternative embodiment, the panels may be of equivalent size.

In conventional adjustable illuminators, the panels are equally sized by width and length and are typically driven at the same power level. The panels are further joined at their edges. Owing to this construction, light is not emitted from a “gap” between the light sources. The lack of light emitting from such areas, together with the uniform supply of power to the panels, can cause optical “dead space” in certain portions of the target treatment area. These portions, in turn, receive less overall light, resulting in a lower dose of treatment in those portions. In some instances, the dose of treatment can be lowered by as much as a factor of five when compared with those areas receiving a desired amount of light.

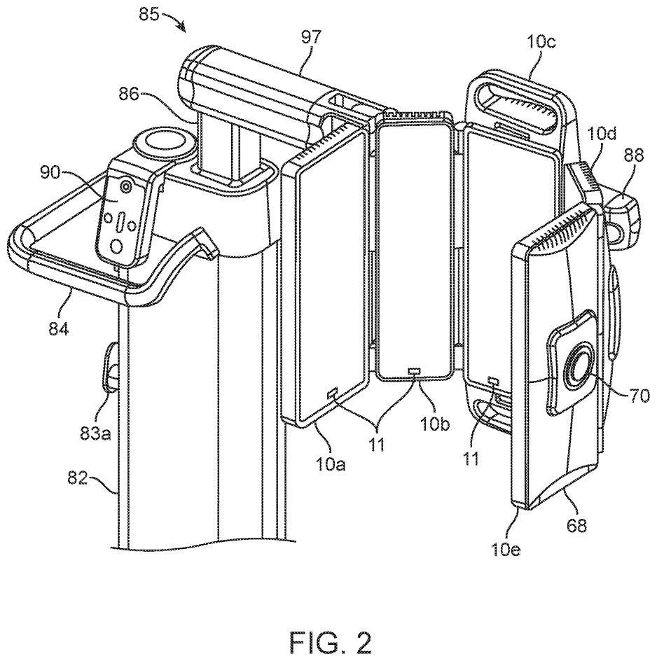

At least one embodiment of the present disclosure includes a plurality of panels 10, wherein at least one panel 10 is of a different width than the other panels. This panel is positioned between two other panels and, in a way, acts as a “lighted hinge” to provide enough “fill-in” light to reduce or eliminate the optical dead spaces when the panels are bent into a certain configuration. Preferably, five panels in total may provide for a desirable increase in the total size of possible treatment areas. Two of the panels (e.g., panels 10b and 10d in FIG. 2) are preferably of a smaller width than the other three larger panels (e.g., panels 10a, 10c and 10e in FIG. 2). The panels are positioned in an alternating manner such that each of the smaller-width panels is situated in between two of the three larger panels to allow for both adjustability and increased uniformity.

In at least one embodiment, each panel 10 contains an array of light emitting diodes (LEDs) 60, which may be configured in an evenly or unevenly spaced pattern across a face of the panel 10. In at least one embodiment, three adjacent panels may be illuminated (e.g., the three inner panels) and two panels may be non-illuminated for a given period, such that illumination may be carried out with a small number of panels than the total number of panels present in the illuminator 100. The number of individual LEDs arranged in a given array is not particularly limited. The panels 10 are configured to uniformly illuminate a treatment surface of a patient via the LEDs. Thus, a substantially homogenous distribution of light may be imparted to the treatment surface.

Preferably, the LEDs may be distributed across a plurality of arrays, where each array of LEDs 60 extends as far to the edges of the panels 10 as possible. In addition, the arrays of LEDs 60 are preferably dimensioned to provide an overall lighted area for a given treatment area based on a range from the 5th percentile of corresponding sizes of female subjects to the 95th percentile of corresponding sizes of male subjects for that particular treatment area. The LEDs 60 emit light at an appropriate wavelength according to the intended treatment or to activate the particular photoactivatable agent used in treatment or diagnosis. For example, when ALA is used as a precursor of a photoactivatable agent for the treatment of AK, the LEDs 60 preferably emit blue light having wavelengths at or above 400 nanometers (nm), for example, about 430 nm, about 420 nm or, for example, 417 nm. However, the LEDs 60 may also emit visible light in other ranges of the spectrum, such as in the green and/or red ranges between 400 and 700 nm, for example, about 625 nm to 640 nm or, for example, 635 nm. For example, the LEDs 60 may also emit light having wavelengths of 510 nm, 540 nm, 575 nm, 630 nm, or 635 nm. In addition, the LEDs 60 may be configured to emit light continuously or may be configured to flash the diodes on and off based on a predetermined interval. Furthermore, the LEDs 60 may be configured such that only one wavelength of light (e.g., blue) is emitted. Alternatively, the LEDs 60 may be configured such that two or more wavelengths of light are emitted from the arrays. For example, the LEDs 60 may be configured to alternately emit blue light and red light.

In at least one embodiment, the LEDs 60 on each of the panels 10 are individually configurable to provide specific power output to certain areas on the panels 10 to compensate for decreased uniformity. For example, the power outputted to each individual diode in an array of LEDs 60 may be individually adjusted. In more detail, the LED arrays may be divided into three general areas, which may be described as “addressable strings.” The current to each area is adjusted in order to adjust the intensity of light emitting from each of the areas. For example, a higher current may be supplied to a given area associated with a particular string or strings, so that it produces a higher intensity of light than another area associated with another string or other strings. Alternatively or in addition, the LEDs 60 may be operated at a higher power level.

In addition, individually regulating power to the LEDs 60 can contribute to the reduction or elimination of optical dead spaces that may otherwise occur where typical illuminator panels are connected. Specifically, power output and/or the emitted light intensity may be increased close to the edges of arrays of LEDs 60 to compensate for the lack of light emitting from an area where neighboring panels meet or adjoin. The narrower panels 10b, 10d are preferably operated at a higher power level and/or at a higher emitted light intensity compared to the wider panels 10a, 10c, 10e in order to provide additional “fill-in” light. In this manner, the LEDs 60 are controllable such that a higher intensity of light is emitted overall from the edges of the panels 10, which may allow for a reduction in any fall-off effect. Thus, such a configuration provides a more uniform illumination output than one in which power and/or intensity are equal across all panels. In at least one embodiment, the illuminator may be configured to adjust each individual diode present in a given LED array, allowing for further calibration.

In at least one alternative embodiment, other types of light sources may be used, such as fluorescent or halogen lamps, instead of or in addition to LEDs.

### Illuminator Structure and Operation

The illuminator may be arranged with a folding apparatus designed to allow maneuverability and reconfigurability of the illuminator panels. In at least one embodiment, the illuminator is adjustable via a main post. The main post is supported by a hydraulic cylinder according to at least one embodiment. The hydraulic cylinder allows raising and/or lowering with manual force applied by a user's digit(s) and/or hands. The cylinder is optionally outfitted with a valve to lock the cylinder in position. Other constructions may be utilized, e.g., instead of a hydraulic cylinder, a spring may be used.

For example, in at least one embodiment, the illuminator is provided with a vertical post which are optionally provided with one or more supports arms. The one or more support arms are configured to support illuminator panel(s) whose height is adjustable. The post may be pushed down into a body of the illuminator or pulled up out of the illuminator. The illuminator may be provided with one or more air cylinders (with at least one check valve and/or at least one spring) to allow the post to be moved with minimal force (e.g., finger pressure). These aspects allow the illuminator to be readily maneuverable and usable under various conditions.

In at least one embodiment, the illuminator uses a ducting arrangement to draw air in via an air distributor (e.g., a fan) at the back of an illuminator panel and output the air via a J-shaped ducting arrangement such that the air gently flows (for example, a laminar flow or similar even, uniform flow) substantially parallel to the front surface (the surface that emits light) of a panel and substantially tangentially to the skin surface. A controller allows a healthcare provider or the patient to control the fan speed at different speeds (for example, slow and fast) in accordance with an observation of the healthcare provider and/or a desire expressed by the patient. The air may be room temperature air (for example approximately 68° F. to approximately 75° F. or approximately 65° F. to approximately 72° F.).

In particular, whether or not the skin is pre-heated as described above, the flowing air is nonetheless cooler than the treatment surface. The temperature is significantly lower than body temperature, and thus feels cooling to the patient. The air may also be cooled below room temperature, in at least one embodiment. The air flow may provide evaporative cooling that reduces the perception or sensation of pain. The air speed can be, for example, approximately 3 to approximately 6 knots, e.g., approximately 3 knots, approximately 4 knots, approximately 5 knots or approximately 6 knots. Such air distributors may have an air flow rate of about 7.5 CFM to about 12 CFM, about 14 CFM, about 5 CFM to about 15 CFM or about 5 CFM to about 20 CFM. Enhancing patient comfort in this manner may influence the willingness of a patient to complete a course of treatment, among other benefits.

The illuminator may be equipped with a thermal management system having additional components beyond those described above. For example, additional air distributors may direct waste heat away from electronic components to the external environment.

Further, in at least one embodiment, the illuminator can be provided with sensors that detect the size of the treatment area positioned in front of the illuminator. In at least one embodiment, information from the sensors can be used (e.g., by a controller) determine the correct light dosing parameters based on the sensed treatment area. In at least one embodiment, the sensors are configured to detect the adjusted position of the illuminator manually set by the user. The detected position of the illuminator may then be used to indicate the intended treatment area. Appropriate light dosing parameters for the specific treatment area may be provided based on the detected position set by the user.

Referring again to FIGS. 1-4, in at least one embodiment, each panel 10 contains at least one vent 68. The vent 68 may be configured to actively (e.g., push/pull) or passively (e.g., provide a path) expel air from the panel 10. In at least one embodiment, the panel 10 may include a plurality of vents 68. For example, the panel 10 may include a first vent 68 and a second vent 68. The first vent maybe disposed at or proximate to a first end of the panel 10, and the second vent 68 may be disposed at or proximate to a second end of the panel 10. Each panel 10 may also contain at least one fan 70. The fan 70 may be configured to draw air into the panel 10 or to expel air from inside the panel 10. A fan 70 may be disposed on a back side of the panel 10. The fan 70 may be disposed at a central location of the panel 10. In at least one embodiment, a panel 10 includes a plurality of fans 70. For example, panel 10c includes a first fan 70 disposed proximate a first end of the panel 10c and a second fan 70 disposed proximate a second end of the panel 10c.

Each panel 10 may contain a distance sensor 11. The distance sensor 11 may detect a distance between the panel 10 and a treatment surface (an affected area) disposed in front of the panel 10. The distance sensor 11 may be automatically turned on when the associated panel 10 is turned on. Detecting the distance between the panel 10 and the treatment surface can facilitate individual adjustment of each panel 10 such that each panel 10 is at a desired distance from the treatment surface.

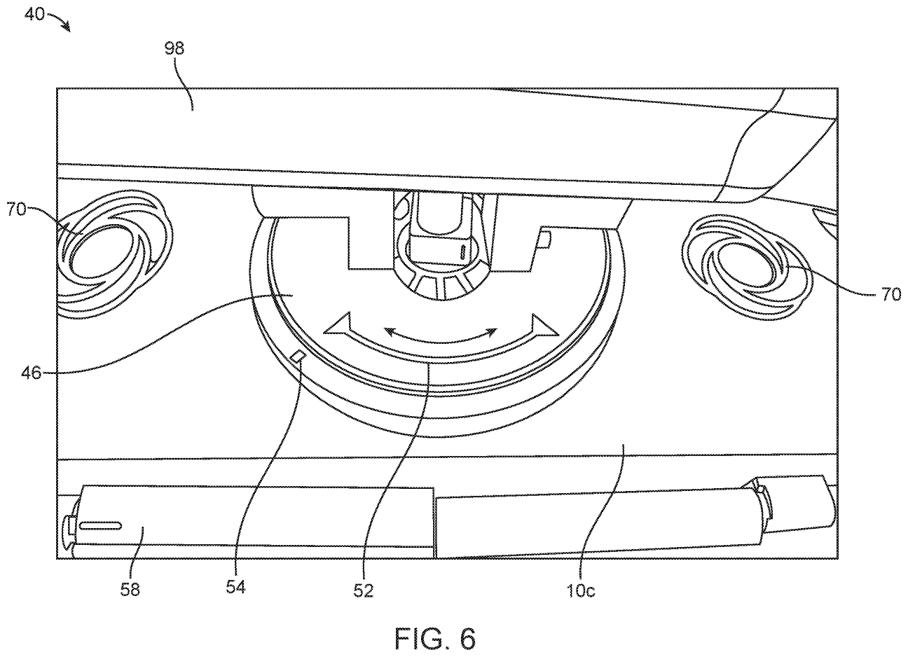

As shown in FIG. 6, in at least one embodiment, the panels 10 may be coupled together via a hinge 58 or other adjustable connection point to facilitate movement of the panels 10 with respect to each other. For example, the panels 10 may be connected in a rotatable manner via nested hinges 58. The illuminator 100 may be configured to fold and unfold via the hinges 58 depending on use. For example, the panels 10 can be in an unfolded (e.g., flat) arrangement when treating areas such as a back, chest or abdomen of a patient. The panels 10 can be in a folded (e.g., U-shaped) arrangement, when treating areas such as a face, scalp, arm, or leg. For example, the outermost panels 10 may face each other (at least in part). The panels 10 can also be in a folded arrangement when in a stowed position. For example, the panels 10 may be wrapped around at least a portion of the illuminator system 105 (e.g., the vertical column 82) when the illuminator 100 is not in use. The hinges 58 may include torque inserts to maintain a position of the panels 10 without using an additional lock. The hinges 58 may also include hard stops that prevent the panels 10 of the illuminator 100 from having an undesired configuration. For example, the hinges 58 may prevent the panels 10 from being fully unfolded (e.g., flat), or from bending beyond the flat configuration (e.g., a U-shape in an opposite direction).

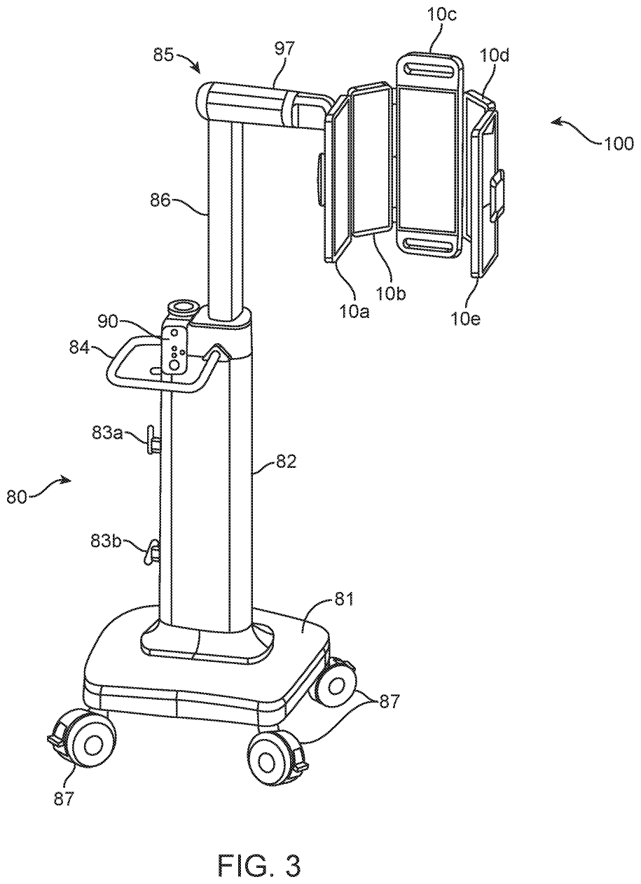

As shown in FIG. 3, the illuminator system 105 may include a moveable stand 80. The movable stand 80 may simplify movement of the illuminator system 105 between various locations and orientations. For example, the moveable stand 80 may include a vertical column 82 coupled to a base 81. The vertical column 82 may extend perpendicular to the base 81. The base 81 may provide support for the vertical column 82 and other components of the illuminator system 105 that are coupled with the vertical column 82. The base 81 may be movable. For example, the base 81 may include a plurality of wheels, shown as casters 87. For example, the base 81 may include four casters 87. The casters 87 may facilitate rolling of the illuminator system 105 from a first location to a second location, or from a first orientation to a second orientation.

As shown in FIG. 3, in at least one embodiment, the illuminator system 105 includes one or more hooks 83. For example, the illuminator may include a first hook 83a and a second hook 83b. The first hook 83a and the second hook 83b may be disposed on a first side of the vertical column 82. The first hook 83a may be disposed above the second hook 83b. The hooks 83 may be rotatably coupled with the vertical column 82. The illuminator system 105 may also include a handle 84 (e.g., a stabilization arm). The handle 84 may be disposed around three sides of the vertical column 82. For example, the handle 84 may extend from a first side of the vertical column 82, wrap around a second side of the vertical column 82, and connect to the vertical column 82 via a third side of the vertical column 82. Apart from a first and second connection point disposed on the first and third side of the vertical column 82, the handle 84 may be spaced apart from the vertical column 82.

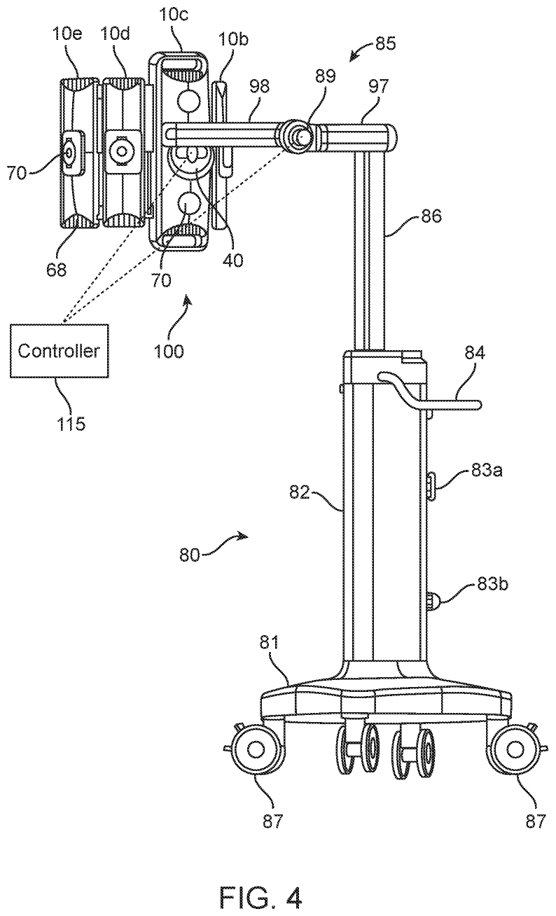

As shown, for example, in FIGS. 1-4, the illuminator system 105 may further comprise an extension member 86. The extension member 86 may be partially disposed within the vertical column 82. The vertical column 82 and the extension member 86 may be configured as a telescoping structure wherein the extension member 86 can extend or slide into the vertical column 82 and extend or slide vertically out of the vertical column 82 between different positions. The extension member 86 may be configured to adjust a height of the illuminator 100. For example, when the extension member 86 is in a retracted position (a low position) and a majority of the extension member 86 is generally disposed in the vertical column 82, as shown in FIGS. 1-2, the illuminator is in a low position. Conversely, when the extension member 86 is in an extended position (an expanded position, or an elevated position) and a majority thereof is disposed generally outside of the vertical column 82, as shown in FIGS. 3-4, the illuminator 100 is in a high (raised or elevated) position. The different positions may be based on a use of the illuminator 100, taking into account one or more of: (i) patient characteristics (e.g., a relatively shorter versus a relatively taller person), (ii) patient orientation (e.g., a standing versus sitting position) or (iii) location of an area to be treated (e.g., the back versus the forearms). The position of the extension member 86 may be adjusted either manually or automatically (e.g., by power). The extension member 86 may be configured to maintain any position between a top position (e.g., fully extended) and a bottom position (e.g., fully retracted).

As seen in FIG. 2, a top of the extension member 86 may be coupled with or be integral with (e.g., for a single component) a connecting arm 85. The connecting arm 85 extends horizontally from the top of the extension member 86. The connecting arm 85 is configured to move with the extension member 86 as the extension member 86 moves relative to the vertical column 82 (e.g., as the extension member 86 moves into or out of the vertical column 82). The connecting arm 85 can include a material of sufficient strength to support other components of the illuminator system 105 (e.g., the illuminator 100).

The connecting arm 85 has a joint, shown as pivot point 89. The pivot point 89 divides the connecting arm 85 into two portions, a first portion and a second portion. The first portion can be a stationary portion 97 and the second portion can be a movable portion 98. The movable portion 98 can extend from an end of the stationary portion 97. The movable portion 98 is rotatably coupled with the stationary portion 97. For example, the movable portion 98 can pivot around the pivot point 89. For example, the movable portion 98 can rotate vertically about the pivot point 89. In at least one embodiment, the movable portion 98 rotates approximately 90 degrees around the pivot point 89 (so as to be rotatable within a range from 0° to approximately 90°).

For example, the stationary portion 97 may define a horizontal plane. The movable portion 98 may rotate between a horizontal position (e.g., parallel with the stationary portion 97 and disposed in the horizontal plate) and a vertically downward position (e.g., perpendicular to the stationary portion 97 and extending downward)). The horizontal position may be a use position or treatment position. The vertically downward position may be a stowed position. In at least one embodiment, the movable portion 98 may rotate approximately up to 180 degrees around the pivot point 89 (e.g., in a range from approximately zero to 180 degrees). For example, movable portion 98 may rotate between the vertically downward position and a vertically upward position (e.g., perpendicular to the stationary portion 97 and extending upward). The vertically upward position may be a use position or treatment position. The movable portion 98 may also be configured to remain at any other angle relative to the stationary portion 97.

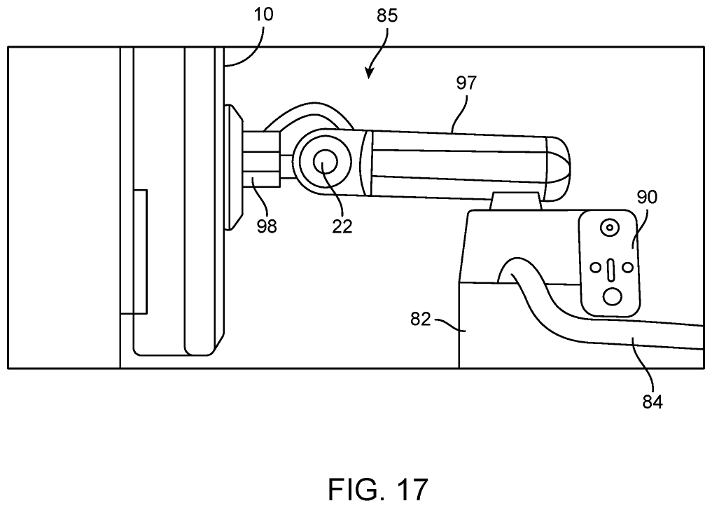

In at least one embodiment, the illuminator system 105 includes an arm lock 22, as shown in FIGS. 1 and 17, among others. The movable portion 98 may be folded into a vertical position, for example, when the illuminator system 105 is not in use or is being stored. The movable portion 98 may be rotated into a horizontal position, for example, when the illuminator system 105 is being used for treatment. The arm lock 22 may be disposed at a location where the movable portion 98 couples with the stationary portion 97. For example, the arm lock 22 may be disposed at the pivot point 89. The pivot point 89 may be disposed approximate to a midpoint of the connecting arm 85 such that the arm lock 22 may be disposed approximate to the midpoint of the connecting arm 85. The arm lock 22 may be activated when no force is applied. To unlock or release the movable portion 98, the arm lock 22 may be depressed. The arm lock 22 may automatically lock the movable portion 98 in a position when the movable portion 98 reaches the use position or the fully stowed position.

The connecting arm 85 is configured to support the illuminator 100 at the various positions described herein. The movable portion 98 is coupled with the illuminator 100 via a mounting mechanism 40, as shown in FIGS. 4-6. The mounting mechanism 40 may include a bracket 42 coupled with the movable portion 98. The bracket 42 defines a first rotational axis, shown as bracket axis 44. The bracket 42 may extend from a first side of the movable portion 98. For example, the bracket 42 may extend from a bottom side of the movable portion 98 when the movable portion 98 is in a horizontal position. The mounting mechanism 40 may further include a plate 46. The plate 46 couples with at least one of the plurality of panels 10 of the illuminator 100. The plate 46 preferably couples with a central panel (e.g., panel 10c) of the illuminator 100. The plate 46 is preferably positioned at a central location of the backside of the panel 10. The plate 46 defines a second rotational axis, shown as plate axis 48. The plate axis 48 maybe perpendicular, or substantially perpendicular, to the bracket axis 44.

In at least one embodiment, the plate 46 includes at least one projection 50. The projection 50 extends from the plate 46 to couple with the bracket 42. In at least one embodiment, the plate 46 includes two projections 50. Coupling the plate 46 with the bracket 42 via the projections 50 facilitates securing of the illuminator 100 to the movable stand 80. The projections 50 are rotatably coupled with the bracket 42 such that the projections 50 and the plate 46 can rotate about the bracket axis 44. With the illuminator 100 coupled with the plate 46, the illuminator 100 can rotate about the bracket axis 44. For example, the illuminator can tilt approximately ±90 degrees from a stowed position (e.g., with the center panel 10 facing the floor). The bracket 42 may utilize one or more torque inserts that are capable of holding the illuminator 100 at any position without an additional lock. The movable portion 98 may remain stationary while the illuminator 100 rotates or tilts.

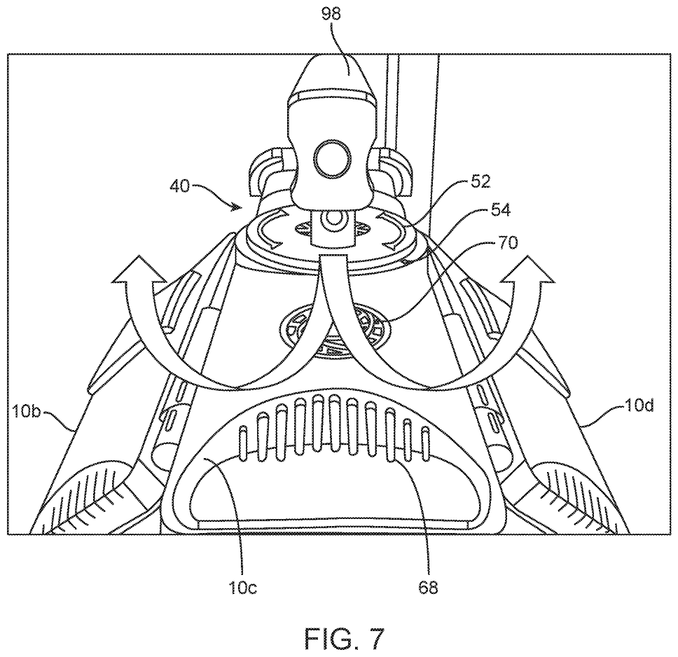

As shown in FIG. 7, the illuminator 100 may rotate or tilt about the bracket axis 44. Rotation about the bracket axis may facilitate placement of the illuminator 100 for treatment. For example, the illuminator 100 may be rotated about the bracket axis 44 such that the illuminator 100 is disposed below the connecting arm 85 (e.g., a stowed or neutral position) with the movable portion 98 in a horizontal position. In such an embodiment, the panels 10 may be facing toward a floor (or ground) where the illuminator system 105 sits. The central panel 10 of the illuminator may be oriented horizontally.

The illuminator 100 may be rotated about the bracket axis 44 such that the illuminator 100 is disposed on a side of the connecting arm 85. For example, the illuminator 100 may be rotated approximately 90 degrees such that the panels 10 face toward a wall. In such an embodiment, the central panel 10 of the illuminator may be oriented vertically. The illuminator 100 can rotate approximately 90 degrees (e.g., in a range from approximately zero degrees up to approximately 90 degrees) to either the left or right side of the movable portion 98, as shown by the arrows of FIG. 7. As such, the illuminator 100 can rotate approximately up to 180 degrees around the bracket axis 44 (e.g., in a range from approximately zero to 180 degrees). The one or more torque inserts can hold the illuminator 100 at any position through the permitted 180 degree range of motion.

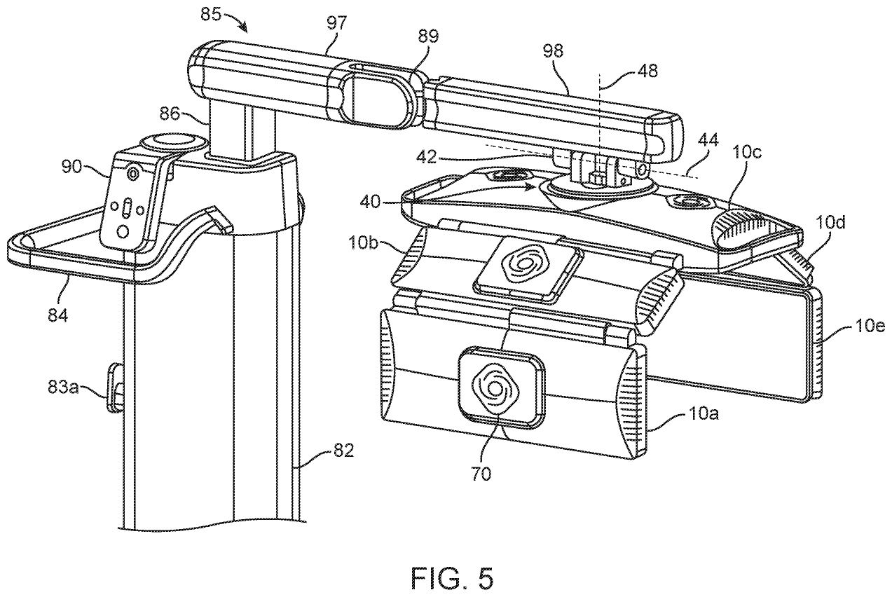

The plate 46 may be rotatably coupled to the panel 10 such that the panel 10, and the other panels 10 of the illuminator 100 can rotate about the plate axis 48 relative to the plate 46. For example, as shown in FIGS. 5 and 6, the illuminator 100 may rotate approximately 90 degrees from a stowed or neutral position (e.g., the center panel 10 is aligned with the stationary portion 97 of the connecting arm 85). In at least one embodiment, the mounting mechanism 40 includes a positioning guide 52 on the plate 46 and a position indicator 54 on the panel 10. The positioning guide 52 indicates the rotation range of the illuminator 100 (e.g., approximately 90 degrees). The position indicator 54 indicates where within the rotation range the illuminator 100 is currently positioned. For example, as shown in FIG. 6, the positioning guide 52 may indicate that the illuminator 100 can rotate as long as the position indicator 54 aligns with a portion of the positioning guide 52. As the illuminator 100 rotates, the position indicator 54 may travel around the positioning guide 52. When the position indicator 54 reaches an end of the positioning guide 52, the mounting mechanism 40 may prevent the illuminator 100 from rotating any further in that direction. For example, the mounting mechanism 40 may have a detent at each end of the positioning guide 52 to lock the illuminator 100 into position. The position of the illuminator 100 may be locked into a position for treatment when the position indicator 54 aligns with an end of the positioning guide 52.

The illuminator 100 can rotate such that the position indicator 54 moves from a first position aligned with a first end of the positioning guide 52 to a second position aligned with a second end of the positioning guide 52. The movement between the first position and the second position can include a rotation of the illuminator 100 of approximately 90 degrees. To move between the first and second positions, the illuminator 100 can move approximately 90 degrees to the left (e.g., clockwise) or 90 degrees to the right (e.g., counter clockwise). The illuminator 100 may also move to any intermediate position disposed between the first and second ends of the positioning guide 52. The illuminator 100 is configured to remain at any desired angle during operation. For example, the position indicator 53 may be disposed between the first and second ends of the positioning guide 52 when the illuminator 100 is being used.

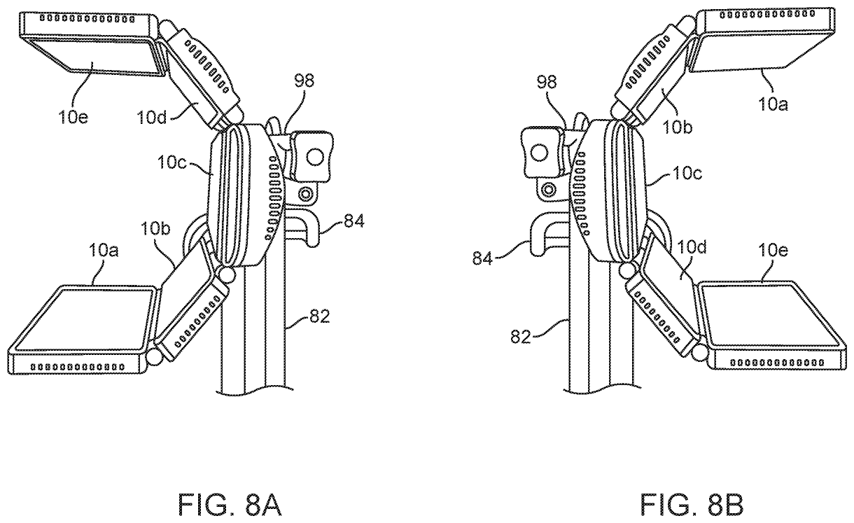

As shown in FIGS. 8A-13, the illuminator 100 may rotate about both the bracket axis 44 and the plate axis 48 regardless of the position of the movable portion 98 of the connecting arm 85 or the height of the extension member 86. For example, in FIG. 8A, the extension member 86 is in a low (retracted) position, the movable portion 98 of the connecting arm 85 is in a horizontal position, the illuminator 100 is rotated about the bracket axis 44 such that the illuminator 100 is disposed on a left side of the connecting arm 85, and the illuminator 100 is rotate such that the panels 10a-10e are disposed in a horizontal orientation. In FIG. 8B, the extension member 86 is the same low position, movable portion 98 is in the same horizontal position, and the panels 10a-10e are still oriented in the same horizontal orientation, but the illuminator 100 is rotated about the bracket axis 44 in the opposite direction such that the illuminator 100 is disposed on a right side of the connecting arm 85. For example, the illuminator 100 rotated under the connecting arm 85 to switch from the left side to the right side. The same movements can be made when the extension member 86 is extended from, and not fully disposed in, the vertical column 82.

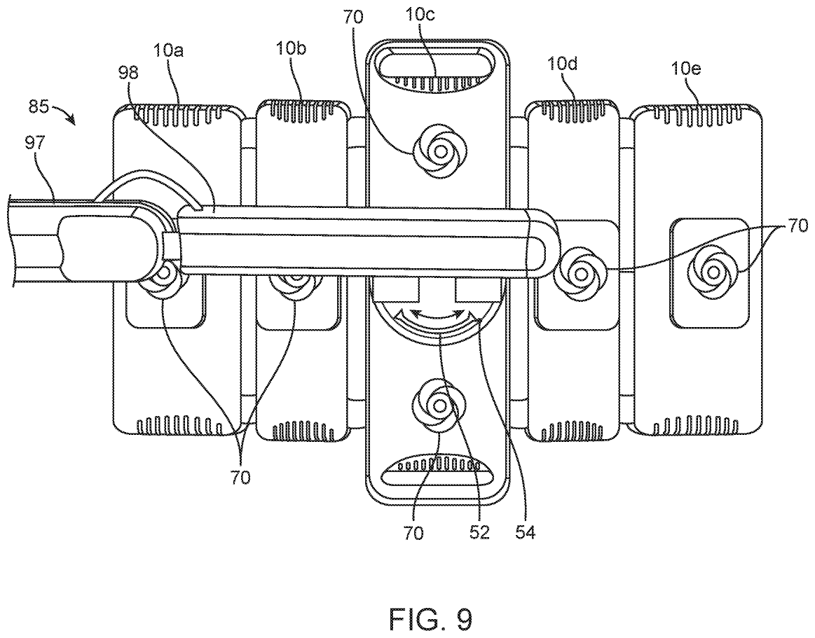

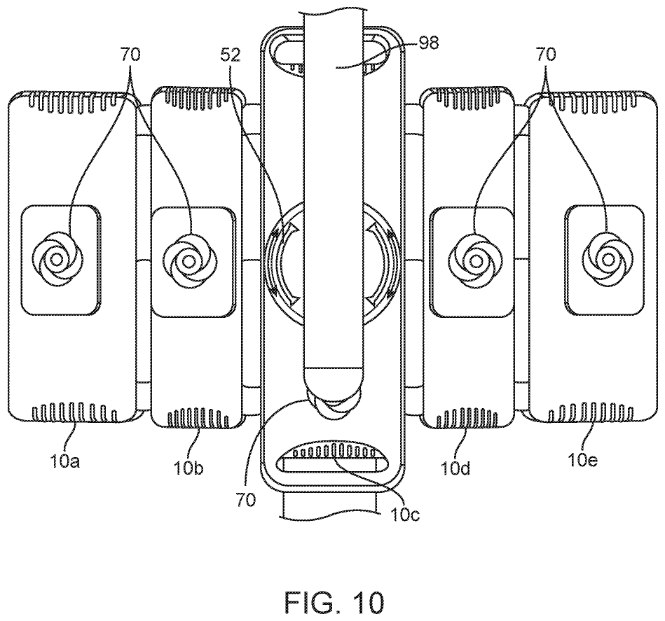

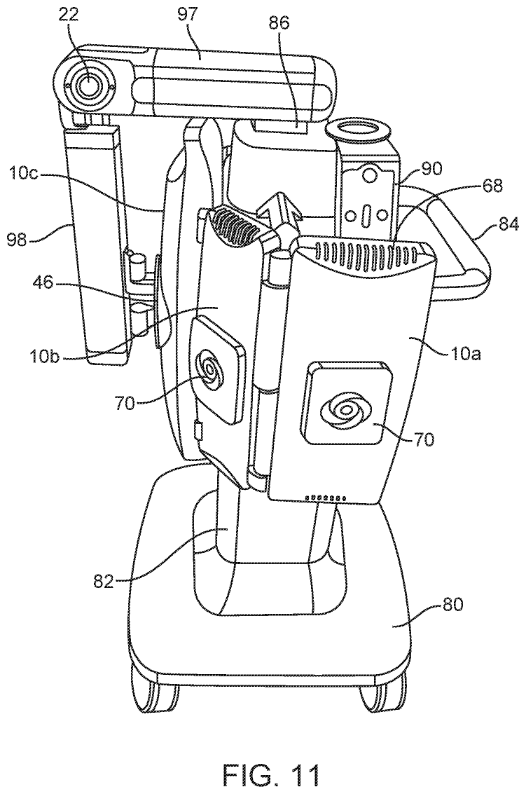

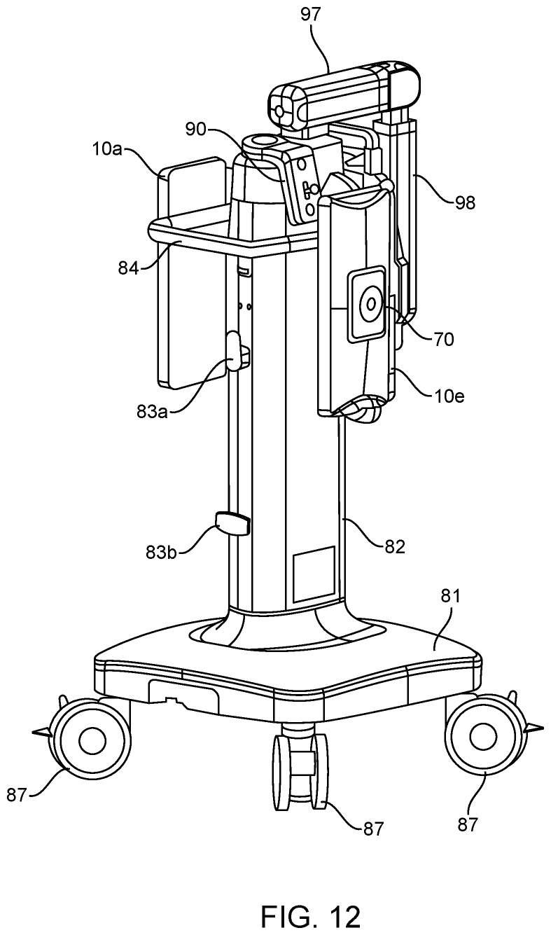

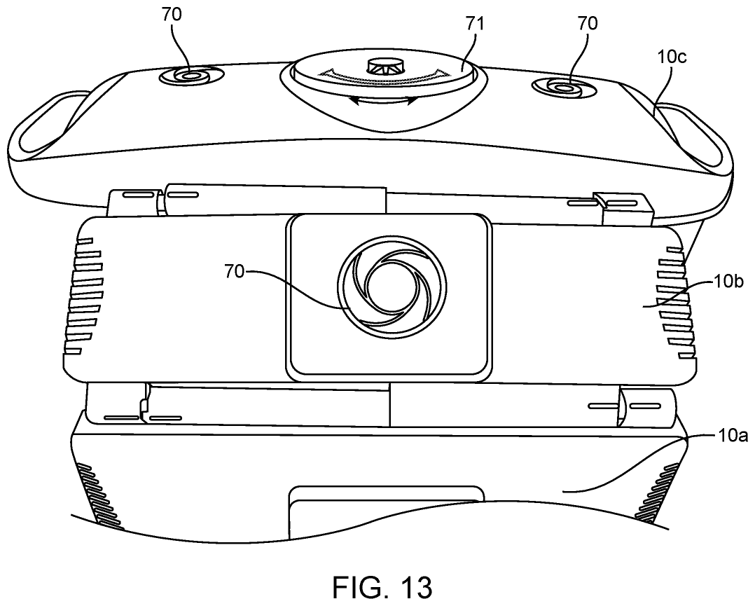

In FIG. 9, the illuminator 100 is still disposed on the right side of the connecting arm 85 (as viewed from the right of the figure), but the illuminator 100 is rotated about the plate axis 48 such that the panels 10a-10e are disposed in a vertical orientation. In FIG. 10, the movable portion 98 is oriented vertically and the illuminator 100 may still rotate about either the plate axis 48 or the bracket axis 44 to a desired orientation. The orientation of the panels 10 with respect to each other can also be modified in any position. For example, with the movable portion 98 in the vertical position, the panels 10 may move between a flat configuration and a folded configuration. For example, FIG. 10 shows the movable portion 98 in a vertical position with the panels 10 in a flat configuration. FIGS. 11-12 show the movable portion 98 still in the vertical position, but with the panels 10 in a folded configuration. FIG. 13 shows the movable portion 98 in a horizontal position with the panels 10 in a folded configuration.

The components of the illuminator system 105 described herein facilitate movement of the illuminator 100 to provide uniform light to a desired treatment area. The illuminator 100 can move between a fully stowed position and various operational positions, and various intermediate positions in between. In the fully stowed position, (i) the extension member 86 is in its lowest position (e.g., a majority of the extension member 86 is disposed in the vertical column 82), (ii) the movable portion 98 of the connecting arm 85 is in a vertically downward position (perpendicular to the stationary portion 97), and (iii) the central panel 10 of the illuminator 100 is aligned with the movable portion 98 (e.g., vertical and at the neutral position relative to both the bracket axis 44 and the plate axis 48). Further, when the illuminator 100 is fully stowed, the panels 10 of the illuminator 100 are configured to be maintained in a U-shaped arrangement and to surround at least a portion of the vertical column 82. The panels 10 of the illuminator 100 are foldable within the contours of the base 81 and the vertical column 82.

In at least one embodiment, an operational position includes the extension member 86 extending at least partially from the vertical column 82 (the vertical column lock 21 can lock the vertical column 82 at any height), the movable portion 98 of the connecting arm 85 being horizontal and parallel with the stationary portion 97, the illuminator 100 disposed at any orientation relative to the bracket axis 44 and the plate axis 48, and the panels 10 of the illuminator 100 in any configuration to provide light to the desired treatment area. Regarding the plate axis 48, the illuminator 100 may be substantially parallel with the movable portion 98 of the connecting arm 85 or may rotate approximately 90 degrees to be substantially perpendicular to the movable portion 98. The illuminator 100 may also be at any angle between 0 and 90 degrees with respect to the plate axis 48. Regarding the bracket axis 44, the illuminator 100 may be in the neutral position and be in the same vertical plane as the stationary portion 97 of the connecting arm 85, or may either (i) rotate up to approximately 90 degrees (e.g., in a range from approximately zero to 90 degrees) in a first direction to be disposed on a first side of the movable portion 98 (out of the plane of the stationary portion 97) or (ii) rotate up to approximately 90 degrees (e.g., in a range from approximately zero to 90 degrees) in a second direction to be disposed on a second side of the movable portion 98 (also out of the plane of the stationary portion 97).

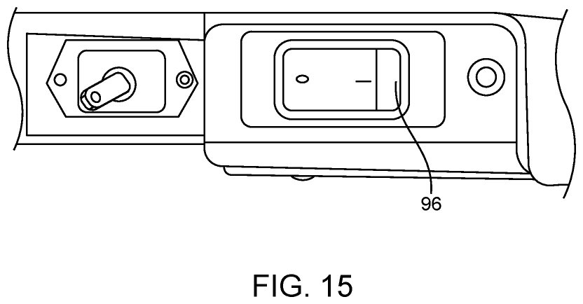

Referring now to FIG. 15, illuminator system 105 includes a main power switch 96. The main power switch 96 may control when power is supplied to the illuminator system 105. The main power switch 96 may be a two-position rocker switch that can toggle between two positions. For example, a first position can activate (e.g., turn on power to) the illuminator system 105 and the second position can deactivate (e.g., disconnect all electrical components of) the illuminator system 105. The first position can place the illuminator system 105 in a stand-by mode. At least one emitter (e.g., an LED 60) may be disposed adjacent to the main power switch 96. The emitter is configured to provide illumination when the illuminator system 105 is in the stand-by mode. The main power switch 96 may be located on the base 81 adjacent to a socket for a removable power cord (e.g., a medical grade power cord).

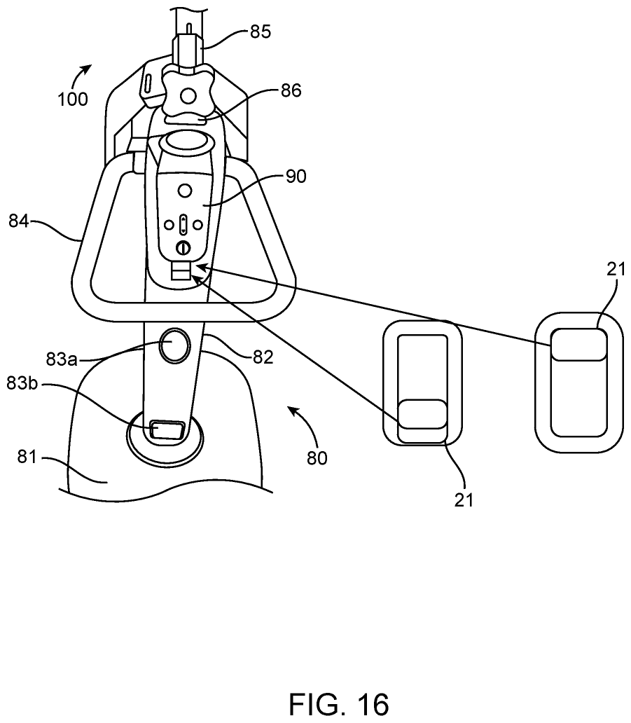

As shown in FIG. 16, the illuminator system 105 includes a vertical column lock 21. The vertical column lock 21 may be disposed below the handle 84. The vertical column lock 21 may be moved between a first position and a second position. The first position may be an up position that unlocks the vertical column 82 such that the extension member 86 can move in and out of the vertical column 82 and the height of the illuminator 100 can be adjusted. A bezel adjacent to the vertical column lock 21 may be a first predetermined color (e.g., green or another color) when the vertical column lock 21 is in the up position to indicate the vertical column 82 is unlocked. The second position may be a down position that locks the vertical column 82 such that the extension member 86 is fixed at its current position. The bezel adjacent to the vertical column lock 21 may be a second predetermined color (e.g., red or another color) when the vertical column lock 21 is in the down position to indicate the vertical column 82 is locked.

### Illuminator Sensor Configuration and Interface

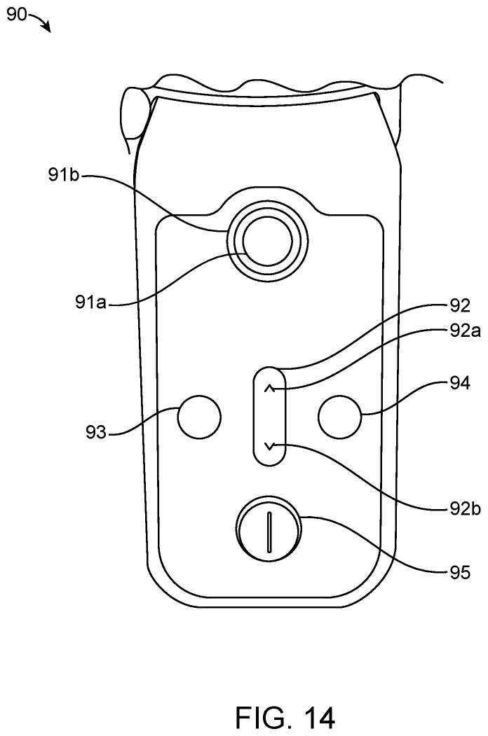

As shown in FIGS. 1 and 14, the illuminator system 105 may include an interface panel 90. The interface panel 90 may be supported by the vertical column 82. The interface panel 90 may be coupled with the vertical column 82 and may be removable from the vertical column 82. The interface panel 90 may include any number of buttons, switches, or other control mechanisms that control aspects of the illuminator system 105. For example, the interface panel 90 may include a power button 91a and a status indicator 91b. The power button 91a may control a setting of the illuminator system 105. For example, the power button 91a may load one of two pre-programmed treatment cycles to the illuminator system 105. For example, the last treatment cycle used (e.g., 10 mW or 20 mW) may be loaded and a time is displayed (e.g., 16:40 or 8:20).

The status indicator 91b may indicate a status of the illuminator system 105. For example, different colors or frequencies of the status indicator 91b may have different meanings. For example, a first color (e.g., blue) may indicate a first status (e.g., normal operation) and a second color (e.g., amber) may indicate a second status (e.g., fault condition). A flashing first color may indicate a third status and a solid first color may indicate a fourth status. Actuating the power button 91a (e.g., pressing the button) at predetermined times may cause predetermined actions. For example, pressing the power button 91a while a treatment cycle is “paused” may cancel the treatment cycle to clear the time displayed. Pressing the power button 91a with a main power switch 96 (described in more detail below) activated may toggle to load or clear a treatment cycle for the illuminator system 105.

The status indicator 91b may include an array of LEDs disposed around the power button 91a (e.g., in an annular pattern or a different pattern). The status indicator 91b displays a status of the illuminator system 105. In at least one embodiment, at a beginning of a treatment, the status indicator 91b may illuminate a steady blue (or another color) to indicate that control electronics of the illuminator system 105 are functioning normally and that associated software is ready for use. The status indicator 91b may change from the steady blue to a slow flashing blue (or another color) when the illuminator system 105 is placed into a pause mode. In at least one embodiment, another color or other colors may be used. The status indicator 91b may return to a steady blue when the cycle resumes. The status indicator 91b may illuminate a steady amber or flashing amber (or another color) if a fault condition is detected (i.e., in response to detecting a fault).

The interface panel 90 may include time adjuster 92. For example, the time adjuster 92 may be a button configured to control a treatment time of the panels 10 (e.g., the time the illuminator is activated). A maximum treatment time may be predetermined. For example, a maximum treatment time may be set at thirty minutes. The time adjuster 92 may be used to adjust an exposure time manually or automatically turn off the LEDs of the panels 10 after a set exposure time has lapsed. The time adjuster 92 may include an up button 92a and a down button 92b to increase or decrease the time, respectively. When first depressed, the up button 92a and the down button 92b change a displayed reading relatively slowly (e.g., within a first predetermined time period, such as over fifteen seconds, etc.). When the up button 92a or the down button 92b remain depressed, the displayed reading changes more quickly (e.g., within a second predetermined time period that is shorter than the first time period, e.g., within five seconds). Depressing and releasing the up and down buttons 92a, 92b quickly allows for an adjustment to the displayed time. For example, each depression may adjust the time by a given interval (e.g., by one second).

The interface panel 90 may include a level adjuster 93. For example, the level adjuster 93 may be a button configured to adjust an intensity or power setting (e.g., power level) of the illuminator 100. For example, the level adjuster 93 may switch the power between two settings (e.g., 10 mW and 20 mW). The power level may be selected after pressing the power button and status indicator 91 to load one of the pre-programmed cycles. The time adjuster 92 may automatically set the correct time for the power level selected. The power level selected may be displayed above the level adjuster 93. For example, the 10 mW setting may be displayed as “10” and the 20 mW setting may be displayed as “20.”

The interface panel 90 may include a comfort adjuster (comfort controller, patient settings controller) 94. For example, the comfort adjuster 94 may be a button or other interface configured to control a patient comfort fan. For example, the comfort adjuster 94 may be a button or other interface configured to switch a setting of a fan between off, low, and high. The patient comfort fan may be controllable by the healthcare provider or patient upon pressing the power button and status indicator 91 to load the treatment cycle. The comfort adjuster 94 can be used to cycle through the three settings. The patient cooling fans may automatically shut off when a cycle timer reaches zero during treatment.

The interface panel 90 may include a start/stop button 95. For example, the start/stop button may be configured to initiate the programmed treatment cycle, pause an active treatment cycle, or restart a paused treatment cycle. Pressing the start/stop button 95 while the treatment cycle is active may cause one or more of the following: (i) pausing or cessation of the treatment cycle, (ii) switching off of LEDs, and (iii) terminating a count-down performed by the timer. The power button and status indicator 91 may flash a predetermined color to indicate that the system is paused. The illuminator system 105 may automatically return to stand-by mode when left paused for a predetermined time or more. For example, the illuminator system 105 may automatically return to stand-by mode if left paused for over 5 minutes. Pressing the start/stop button 95 while the system is paused may cause the treatment cycle to resume, the LEDs to illuminate, and the timer to resume counting down. The power button and status indicator 91 may display a steady predetermined color to indicate normal operating status (e.g., a steady blue color, or another color).

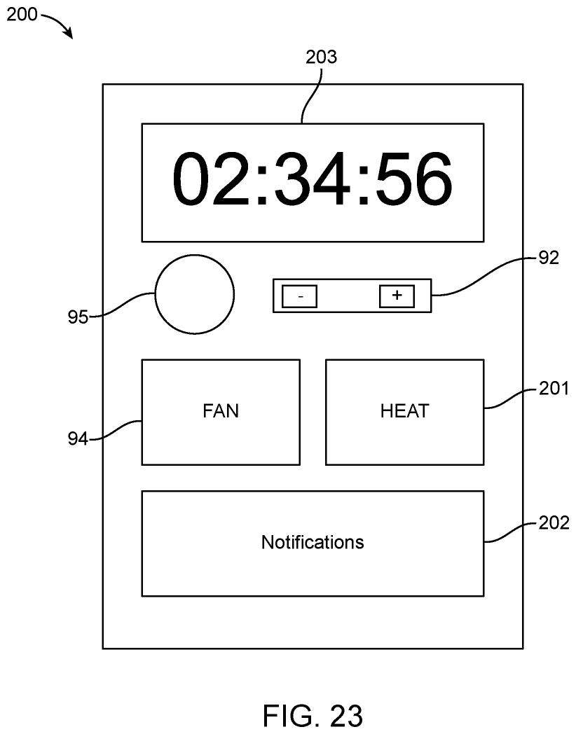

As shown in FIG. 23, the illuminator system 105 may include a touch screen 200. The touch screen 200 may be a part of the interface panel 90, the controller 115, or some other independent device (e.g., a user device). The touch screen 200 may allow a user to control, monitor, and adjust settings of the illuminator system 105. For example, the touch screen 200 may include at least one of the power button 91a, the status indicator 91b, the time adjuster 92, the lever adjuster 93, the comfort adjuster 94, and/or the start/stop button 95. The touch screen 200 may include additional features (e.g., buttons, displays, notifications, etc.) for a user to monitor and control the settings.

For example, the touch screen 200 may provide a heat controller 201 to control the heat directed to the patient for pain management. The touch screen 200 may provide a notification window 202 to provide notifications or alerts to the user. The touch screen 200 may provide a time indicator 203. The time indicator 203 may display a time remaining for the treatment. The time displayed on the time indicator 203 may change as time progresses and may change as adjusted via the time adjuster 92. In at least one embodiment, the remaining exposure time is displayed in minutes and seconds. Prior to pushing the start/stop button 95, the exposure time indicator displays the amount of exposure time set. When the start/stop button 95 is pressed, the exposure time indicator 203 counts down the amount of exposure time remaining. The exposure time indicator 203 may turn off automatically when the display reaches zero. The interface panel 90 may include some or all of these additional features, even if the interface panel 90 does not include the touch screen 200.

The illuminator system 105 may include one or more sensors 110. In at least one embodiment, the illuminator system 105 may include a sensor 110 configured to detect a size of a treatment area of a patient. In at least one embodiment, the illuminator system 105 may include a sensor 110 configured to detect a position of the illuminator 100. The position of the illuminator 100 may include the height of the extension member 86, the orientation of the illuminator 100 (e.g., vertical, horizontal, to the right or left of the connecting arm 85), or the configuration of the panels 10 (e.g., U-shaped, flat, etc.). The position of the illuminator 100 and the size of the treatment area can be used to determine the correct light dosing parameters for the treatment. The sensor 110 can be disposed on any component of the illuminator system 105. For example, a sensor 110 may be disposed on the vertical column 82, a panel 10, and/or the connecting arm 85, among others. The sensor 110 can be any type of sensor configured to detect data indicative of the position of the illuminator.

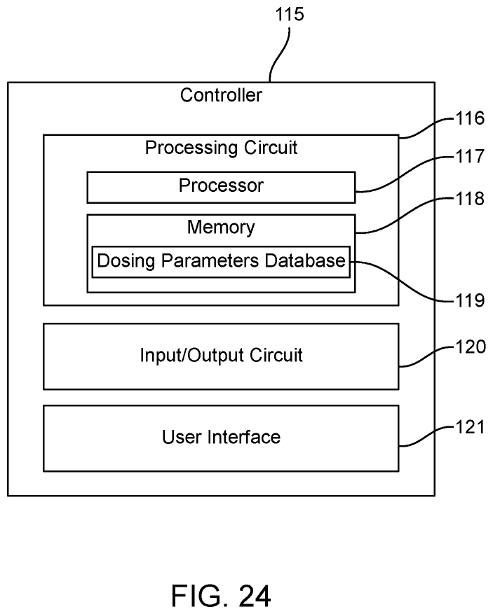

The illuminator system 105 may include a controller 115. As shown in FIG. 24, the controller 115 may be configured to monitor and control various components of the illuminator system 105. For example, the controller 115 may be configured to control the heat source currents to accommodate differing tissue geometries and to provide differing power levels, including varying the current over time to modulate a patient's pain tolerance. The variation in current may be in response to input from sensors such as a distance sensor or a sensor that indicates the relative position of the panels.

In at least one embodiment, the controller 115 may also be configured to transition the illuminator system 105 between a curved geometry and a flat geometry and maintain uniformity and power throughout the transition. The controller 115 may also include a processing circuit 116. The processing circuit 116 may include a processor 117 and a memory 118. The memory 118 (e.g., storage device) may include one or more devices (e.g., RAM, EPROM, optical disk storage, magnetic disk storage flash memory, hard disk storage, or any other medium) for storing data and/or computer code for completing or facilitating the various processes and functions described in the present disclosure. The memory 118 may be or include transitory memory or non-transitory memory, and may include any type of information structure for supporting the various activities and information structures described in the present disclosure.

According to at least one embodiment, the memory 118 is communicably connected with the processor 117 and includes computer code for executing (e.g., by the processor 117) the processes described herein. The processor 117 may be a general purpose single-chip or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor or any typical processor, controller, microcontroller, or state machine. The processor 117 may also be implemented as a combination of computing devices, such as a combination of a DSP and a microprocessor, a plurality of microprocessors, one or more microprocessors in conjunction with a DSP core, or any other such configuration. In at least one embodiment, particular processes and methods may be performed by circuitry designed for a given function.

In at least one embodiment, the memory 118 may include a dosing parameter database 119. The dosing parameter database 119 may include relationships between dosing quantities (including exposure time), light intensities, distances between the panels and the treatment surface, and size of the treatment surface, among other data associated with treatment via the illuminator system 105. For example, a specific duration of exposure to light from the illuminator 100 can be associated with a specific light intensity, a specific distance between the panels and the treatment surface, and a specific size of the target surface. The processor 117 can use the data stored in the dosing parameter database 119 to determine the appropriate dosing parameters for a treatment session for a patient.

In at least one embodiment, the controller 115 may include an input/output (I/O) circuit 120. The I/O circuit 120 may be configured to receive signals or data from external devices and transmit signals or data to external devices.

In at least one embodiment, the controller 115 may include a user interface 121. The user interface 121 may be a touch screen. The user interface 121 may be configured to display the information received by the controller 115 via the I/O circuit 120 or retrieved from the memory 118. The user interface 121 may be configured to receive input from a user. For example, the user interface 121 may have interactive sections with which a user can interact with and provide information. The interactive sections may be buttons, switches, or input fields, among others. The controller 115 may receive the user input via the I/O circuit 120 and may be configured to store the user input in the memory 118 or use the user input to generate an output. For example, the user interface 121 may be configured to provide a display that shows the orientation and position of the illuminator 100 based on information received from the distance sensors 11 and the sensors 110. For example, when the illuminator 100 is in a use configuration, the user interface 112 may show the illuminator 100 in its current position. The display may include a current distance of each panel 10 from the treatment area detected by the distance sensors 11.

In at least one embodiment, the controller 115 may be communicably coupled with one or more components of the illuminator system 105. For example, the controller 115 may be communicably coupled with a distance sensor 11. The distance sensor 11 may be configured to transmit a signal to the controller 115 indicative of the distance between the panel 10 and the treatment area. The controller 115 may be communicably coupled with a sensor 110. The sensor 110 may be configured to transmit a signal to the controller 115 indicative of the position or orientation of the illuminator 100. The controller 115 may be communicably coupled with the mounting mechanism 40. The controller 115 may be configured to transmit a command to the mounting mechanism 40 to orient the illuminator 100 in a desired position. The controller 115 may be communicably coupled with the panels 10 of the illuminator 100. The controller 115 may be configured to transmit a command to the panels 10 to orient the panels 10 in a desired configuration (e.g., U-shaped). The controller 115 may be communicably coupled with the components via a wired or wireless connection.

### Illuminator Cooling System and Method

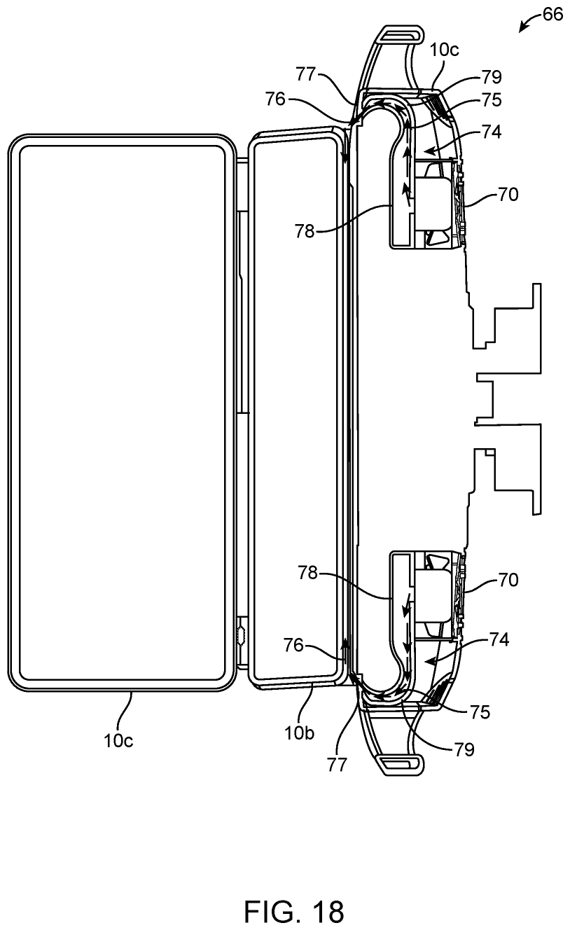

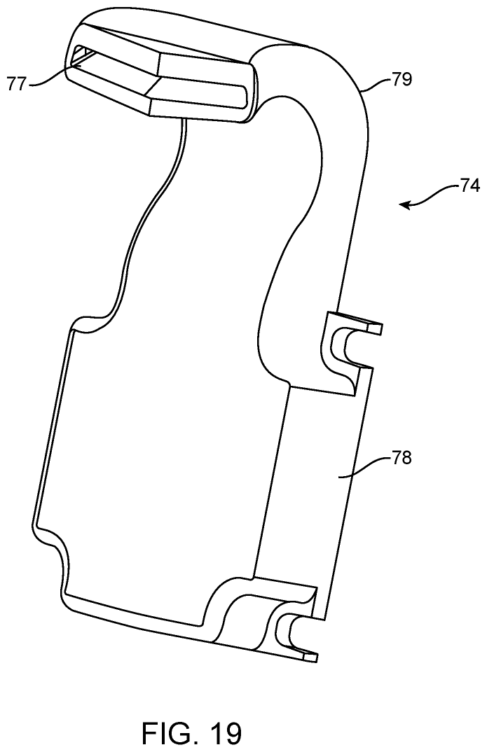

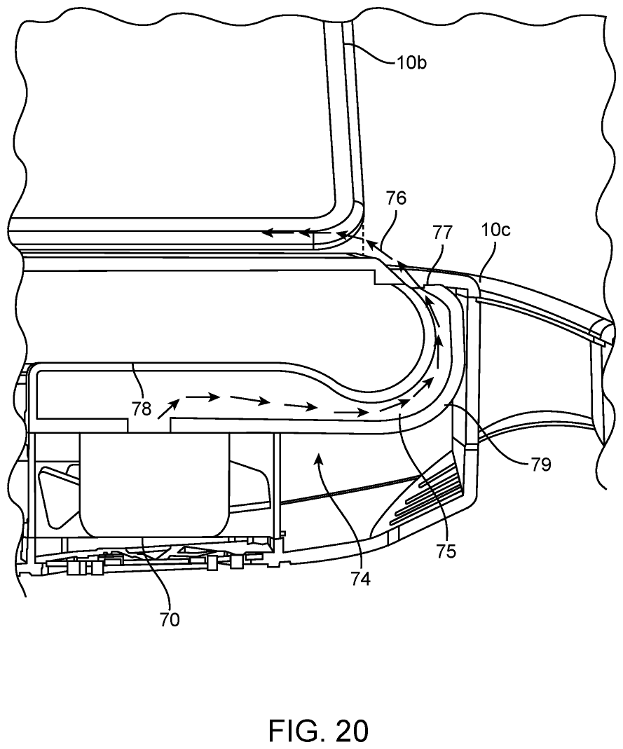

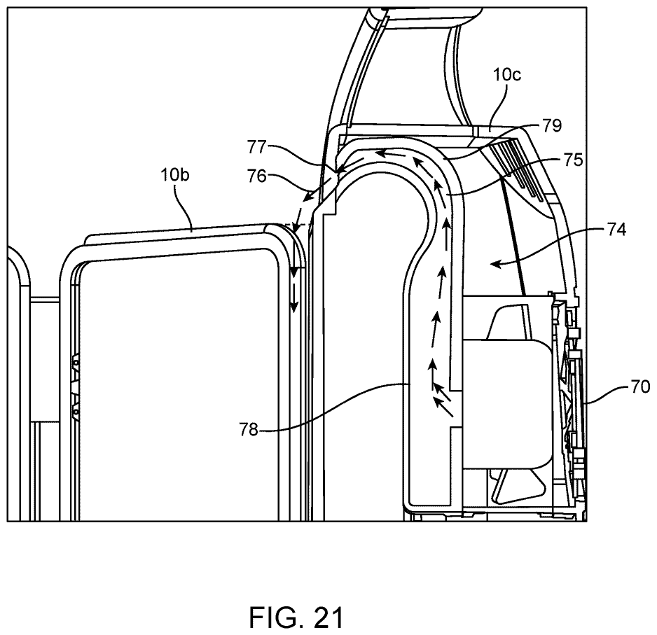

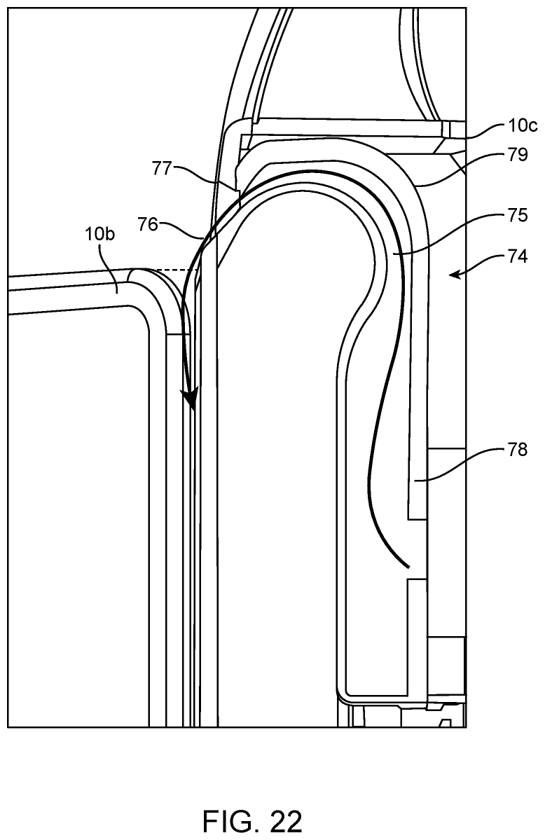

As shown in FIGS. 18-23, at least one of the panels 10 of the illuminator system 105 (e.g., panel 10c) may include a patient cooling fan system 66. The patient cooling fan system 66 may be configured to blow air across a surface of the panel 10 such that the air is tangential to a patient's skin to provide a soothing effect to the patient. The patient cooling fan system 66 includes a fan plenum 74. The fan plenum 74 may define a serpentine path for air to flow, shown as air path 75. For example, the fan plenum 74 may include a body 78 and a neck 79. The body 78 defines a cavity for receiving the air. The cavity can have a first thickness. The body 78 transitions to the neck 79 that has a second thickness. The first thickness is larger than the second thickness. The neck 79 can define a serpentine air path 75 for the airflow 76 until the air reaches the plenum outlet 77.

In at least one embodiment, the fan plenum 74 may be disposed within the panel 10. The patient cooling fan system 66 may include a fan 70. The fan 70 may be configured to draw air in from the environment and push the air into the fan plenum 74. The fan 70 can push the air through the air path 75 of the fan plenum 74 to a plenum outlet 77. The air path 75 and the plenum outlet 77 are configured to generate an airflow 76 that moves parallel, or substantially parallel, to the face of the panel 10.

In at least one embodiment, the panel 10 may include a plurality of fan plenums 74. For example, a first fan plenum 74 may be disposed at a first end of the panel 10 and a second fan plenum 74 may be disposed at a second end of the panel 10. The first and second fan plenums 74 may generate an airflow 76 that is parallel, or substantially parallel, to the face of the panel 10, but a first airflow 76 from the first fan plenum 74 may be directed in a first direction and a second airflow 76 from the second fan plenum 74 may be directed in a second direction. The second direction may be opposite the first direction. For example, the first airflow 76 may move down the face of the panel 10 and the second airflow 76 may move up the face of the panel 10.

In at least one embodiment, the heat source may be provided separately from the illuminator 100 or integrated therein. In at least one embodiment, the heat source (a thermal delivery device) may be an infrared (IR) quartz heater. In at least one embodiment, the heat source may comprise frame mounted resistance tape heaters or a plurality of heaters, including at least one selected from the group including IR LEDs, resistance cartridge heaters, positive temperature coefficient heaters, or IR quartz heaters, as mentioned above. The heat may be deliberately generated and directed towards the area to be treated, as opposed to ambient heat in the clinical setting or byproduct heat from one or more operating mechanisms of the illuminator. In at least one embodiment, the heat is intentionally generated and directed toward the patient and the patient is still further heated by ambient and/or byproduct heat. In at least one embodiment, the heat is administered in the form of a heat mask, such as a sodium acetate mask configured to heat upon crystallization. In at least one embodiment, the heat source is a heating pad.

Thus, components or operating mechanisms of the illuminator can be configured to generate heat that can be deliberately targeted toward the patient. For example, the illuminator may include one or more fans that draw air across such components or mechanisms to deliver heat to the patient. The heat may alleviate pain or discomfort experienced by the patient. And, as noted above, heating additionally accelerates the conversion of ALA to porphyrin.

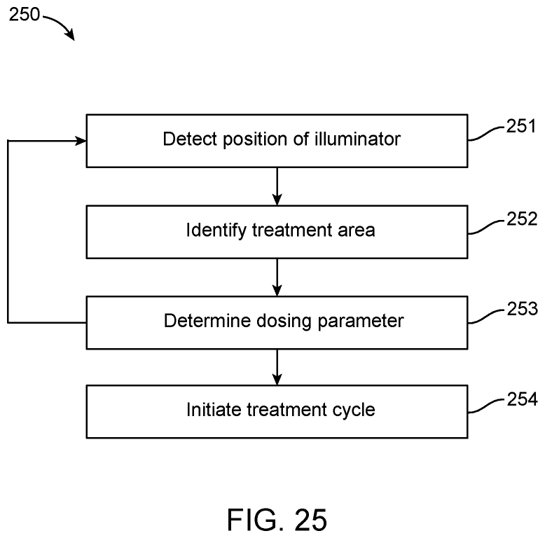

Referring now to FIG. 25, a method 250 of providing photodynamic therapy is shown, according to an exemplary embodiment. Method 250 may include detecting a position of the illuminator (step 251), identifying a treatment area (step 252), determining a dosing parameter (step 253), and beginning treatment (step 254). In particular, the method can include detecting the position of the illuminator 100, e.g., when the illuminator is in a neutral position or a rotated position of up to 90° relative to an axis.

At step 251, one or more processors and/or sensors may detect a position of an illuminator 100. For example, the controller 115 may receive a signal from a sensor 110 indicating a position of a panel 10 of the illuminator 100. For example, the sensor 110 may be a distance sensor or a sensor that indicates a relative position of the panels 10 of the illuminator 100. The controller 115 may receive a signal from a plurality of sensors 110 indicating a position of a corresponding panel 10. The controller 115 may determine the position of the illuminator 100 based on the plurality of signals. Step 251 may include at least one of detecting a height of an extension member 86, detecting an orientation of the movable portion 98 of the connecting arm 85 (e.g., vertically down, vertically up, horizontal), detecting an orientation of the illuminator 100 (e.g., rotation angle around at least one of the bracket axis 44 and the plate axis 48), and/or detecting an arrangement of the plurality of panels 10 of the illuminator 100 (e.g., U-shaped, flat).

At step 252, one or more processors or sensors may identify characteristics of a treatment area. For example, the controller 115 may identify or infer a location, shape or size of treatment area based on the position of the illuminator 100. For example, the signals received by the controller 115 may indicate that the panels 10 of the illuminator 100 are positioned vertically, in a U-shape, and are disposed at a specific height. Based on data from the signal and/or sensors, the controller 115 may identify that a face of a patient as the treatment area. Identifying the treatment area may also include determining a shape or size of the treatment area. For example, the controller 115 may determine the size of the treatment area based on signals received from the sensors 11, 110. In some embodiments, a panel position sensor may be used to identify characteristics of the treatment area based on, for example, a look up table. In some embodiments, an active optical or ultrasonic sensor could be used to identify characteristics of the treatment area.

In at least one embodiment, the detection of the position of the illuminator and/or the identification of the treatment area, such as its location, shape or size, may be determined in furtherance of enhancing photodynamic therapy provided using the illuminator 100. For example, by using sensors to detect the shape of the surface to be treated, the LED arrays can be individually configured to emit more intense light to only those areas that require it. Additionally, the sensors can be used to detect the orientation of one or more panels (e.g., whether a panel is angled or folded flat) and may be used to configure the LEDs to emit more or less intense light in areas as desired.

In particular, in at least one embodiment, at least one sensor detects an orientation of at least one panel and provides detection information (a detection result) to the controller 115. The sensors may include one or more encoders, such as one or more angle encoders, which are provided at one or more locations on the panels. In at least one embodiment, at least one sensor is a microswitch configured to sense a position of at least one panel. In at least one embodiment, a plurality of sensors may include an encoder, a microswitch, or combinations thereof. The sensors are communicated with the controller 115 and are configured to provide information about the panel orientation, such as an angle at which a panel is disposed, to the controller 115. The controller then controls the intensity of light in accordance with a detection result. In at least one embodiment, a plurality of sensors provides information to the controller so that the controller may carry out a determination as to whether the illuminator has a configuration that is one of a plurality of preset configurations. For example, the controller may store, in a memory, information relating to one or more preset configurations (e.g., for a bent illuminator, a flat illuminator, etc.).

When the controller receives information transmitted from the sensors, the controller may compare the sensed information to the preset configurations to determine a match between the sensed information and one or more preset configurations. The controller may further store a protocol for altering intensity which is executed upon determining a match between the sensed information and the preset configuration. For example, if the illuminator is detected to be in a U-shaped configuration, the controller implements a light intensity output which is correlated to the preset protocol for a U-shaped illuminator. The controller may further compare an existing intensity to an intensity associated with a particular configuration and determine whether the intensity should be adjusted. This allows for an increase in uniformity of light exposure in an efficient manner as power output and/or light intensity is increased to only certain diodes, in accordance with need. In at least one embodiment, a plurality of preset configurations may be presented to a healthcare provider, e.g., on a touch screen, who may then select the preset configuration corresponding to the physical arrangement of the illuminator in the treatment or clinical environment.

At step 253, one or more processors may determine a dosing parameter. For example, the controller 115 may compare the data received from the sensors 11, 110 with the data stored in the dosing parameter database 119. Based on the comparison, the controller 115 may determine a dosing parameter suitable for treatment for a given position of the illuminator 251 and a given treatment area. The dosing parameter may be a duration of exposure or a light intensity, among others.

The one or more processors may detect an adjusted position of the illuminator 100. In such an embodiments, steps 251-253 can be repeated until a final position of the illuminator 100 is established.

At step 254, one or more processors may initiate a treatment cycle. For example, the controller 115 may initiate a treatment cycle based on the determined dosing parameter. The controller 115 may actuate the LEDs 60 of the panels 10 of the illuminator 100 at a specified intensity based on the dosing parameter. The controller 115 may set a treatment duration based on the dosing parameter. The controller 115 may actuate the patient cooling fan system 66.

Thus, the controller allows a healthcare provider to control one or more of the following aspects: (i) the treatment cycle, (ii) LED actuation, (iii) LED intensity, (iv) treatment duration, and (v) cooling (i.e., fan-induced cooling) of the patient (among other aspects of treatment). The healthcare provider may adjust any or all of (i)-(v) throughout treatment using the controller 115. In particular, the controller 115 may be used (e.g., manipulated by a healthcare provider or the patient) to operate the patient cooling fan system 116 to cause cooled air to be directed to the patient. The cooling air may thus be delivered in response to an input from the controller 115 as it is operated during treatment. For example, by providing cooling air to the patient via the patient cooling fan system 116, a sensation of pain or discomfort experienced by the patient may be alleviated.

It should be noted that the term “example” as used herein to describe various embodiments is intended to indicate that such embodiments are possible examples, representations, and/or illustrations of possible embodiments (and such term is not intended to connote that such embodiments are necessarily extraordinary or superlative examples).

As utilized herein, the term “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

While this specification contains specific implementation details, these should not be construed as limitations on the scope of any embodiment or of what may be claimed, but rather as descriptions of features specific to particular implementations of particular embodiments. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. For example, the implementations described herein can be used in conjunction with (and applied to) the compositions, illuminators, devices, dressings, methods of treatment, processes and techniques set forth in the patents and/or patent applications cited above.

The construction and arrangement of the various exemplary embodiments are illustrative only. Recognizing that one or more embodiments have been described in detail in this disclosure, those skilled in the art who review this disclosure will further appreciate that many modifications are possible (e.g., variations in sizes, dimensions, structures, shapes and proportions of the various elements, values of parameters, mounting arrangements, orientations, etc.) without materially departing from the novel teachings and advantages of the subject matter described herein. Other substitutions, modifications, changes and omissions may also be made in the design, operating aspects and arrangement of the various exemplary embodiments without departing from the scope of the instant disclosure.

What is claimed is:

1. A method of performing photodynamic therapy, comprising:
applying, to the skin of a patient, a topical composition including
(a) 5-aminolevulinic acid (ALA) hydrochloride, and
(b) a vehicle comprising at least one chelating agent to enhance accumulation of protoporphyrin IX (PpIX) in the skin;
incubating the topical composition; and
following incubation, applying, to the skin, heat from a heat source for at least a first time period.

2. The method of claim 1, wherein the at least one chelating agent is selected from ethylenediaminetetraacetic acid (EDTA) or a pharmaceutically acceptable salt thereof.

3. The method of claim 1, further comprising:
following incubation, exposing the skin to light from a light source for a second time period, wherein the heat is also applied during the second time period.

4. The method of claim 1, wherein the incubation occurs for between about 2 hours to about 3 hours, and a sum of the first time period and the second time period is about 13 minutes.

5. The method of claim 1, wherein light is not applied to the skin prior to applying the heat.

6. The method of claim 1, wherein applying the heat to the skin for 13 minutes increases an amount of PpIX present in the skin by more than 50%.

7. The method of claim 1, wherein applying the heat to the skin for 13 minutes increases an amount of PpIX present in the skin by more than 80%.

8. The method of claim 1, wherein the aminolevulinic acid hydrochloride is present in an amount of 20% w/w of the topical composition, and the at least one chelating agent is present in an amount of about 0.1% to about 0.15% of the topical composition.

9. A system for photodynamically diagnosing or treating a patient, comprising:
a mobile base;
a vertical column extending perpendicular from the base;
a controller supported by the vertical column;
an extension member disposed at least partially within the vertical column;
an arm mounted to a top of the extension member; and
an illuminator connected to the arm, the extension member and the arm being movable relative to the vertical column to allow adjustment of a position of the illuminator, the illuminator comprising a plurality of panels configured to uniformly illuminate a treatment surface of a patient.

10. The system of claim 9, wherein the plurality of panels comprises five panels configured to form a U-shaped configuration.

11. The system of claim 9, wherein the arm comprises a stationary portion and a moveable portion, and the illuminator is configured to move between a stored position and an operational position, wherein, in the stored position, the illuminator is configured such that:
the extension member is in a retracted position;
the movable member is disposed in a vertically downward position; and
the plurality of panels define a U-shaped configuration and are arranged at least partially around the vertical column.

12. The system of claim 9, wherein the extension member is configured to slide vertically relative to the vertical column to adjust a height of the illuminator.

13. The system of claim 9, wherein the arm comprises a joint dividing the arm into a stationary portion and a movable portion, wherein the movable portion is configured to rotate up to approximately 90 degrees around the joint to position the illuminator at a desired location.

14. The system of claim 9, further comprising a mounting mechanism to connect the illuminator to the arm, wherein the mounting mechanism defines an axis perpendicular to the vertical column, and the illuminator is configured to rotate approximately up to 180 degrees around the axis.

15. The system of claim 9, further comprising a plate, the plate defined at least in part by an axis that extends through a center of the plate, wherein the illuminator is configured to rotate up to approximately 90 degrees around the axis relative to the plate.

16. The system of claim 9, further comprising a mounting mechanism defining a first axis and a second axis, the first axis being perpendicular to the second axis, wherein the illuminator is configured to rotate about both of the first axis and the second axis.

17. The system of claim 9, wherein the arm comprises a stationary portion and a movable portion, the system further comprising:
a mounting mechanism comprising a bracket and a plate;
a first axis defined by a joint of the arm;
a second axis defined by the bracket; and
a third axis defined by the plate,
wherein the movable portion is configured to rotate up to approximately 90 degrees around the first axis, the illuminator is configured to rotate up to approximately 180 degrees around the second axis, and the illuminator is further configured to rotate approximately 90 degrees around the third axis.

18. The system of claim 9, further comprising a plurality of distance sensors, wherein each of the plurality of distance sensors is configured to detect a distance between a respective panel of the plurality of panels and the treatment surface.

19. The system of claim 9, further comprising a patient cooling fan system, the patient cooling fan system comprising:
a fan plenum disposed in at least one of the plurality of panels, the fan plenum defining an air path;
a plenum outlet disposed at the end of the air path; and
a fan arranged to force air through the fan plenum and the plenum outlet, wherein the air path and the plenum outlet are configured to allow air flow to move substantially parallel to a face of the at least one of the plurality of panels.

20. The system of claim 9, further comprising a patient cooling fan system, the patient cooling fan system comprising:
a first fan plenum disposed in a panel of the plurality of panels, the first fan plenum comprising a first air path and a first plenum outlet, the first plenum outlet disposed proximate to a first end of the panel;
a first fan to force air through the first air path and the first plenum outlet;
a second fan plenum disposed in the panel, the second fan plenum comprising a second air path and a second plenum outlet, the second plenum outlet disposed proximate a first end of the panel; and
a second fan configured to force air through the second air path and the second plenum outlet, wherein the first air path and the first plenum outlet are configured to allow air to flow substantially parallel to a face of the panel in a first direction, and wherein the second air path and the second plenum outlet are configured to allow air to flow substantially parallel to the face of the panel in a second direction, the second direction opposite the first direction.

21. The system of claim 9, further comprising a controller configured to detect a position of the illuminator and to determine a dosing parameter based on the position of the illuminator.

22. The system of claim 9, further comprising an interface panel removably coupled to the vertical column, the interface panel comprising:
a power button configured to control a setting of the system;
a status indicator configured to indicate a status of the system; and
a timer configured to adjust a treatment time for treating the patient.

23. A method of photodynamically diagnosing or treating a patient, comprising:
detecting a position of an illuminator via one or more of a plurality of sensors, the illuminator being rotatable in a range from approximately zero degrees up to approximately 90 degrees relative to a mounting axis thereof;
identifying a treatment surface of the patient;
determining a dosing parameter;
initiating a treatment cycle; and
causing air cooler than the treatment surface to be directed to the patient responsive to an input from a controller which is operable during the treatment cycle to alleviate pain following administration of 5-aminolevulinic acid (ALA) to the patient.

24. The method of claim 23, wherein detecting a position of an illuminator comprises:
detecting an arrangement of a plurality of panels of the illuminator.

25. The method of claim 23, further comprising:
heating the treatment surface via a heat source.

26. An illuminator for diagnosing or treating a patient, comprising:
a plurality of panels; and
a plurality of distance sensors, wherein each of the distance sensors is associated with a corresponding panel of the plurality of panels,
the illuminator being configured to rotate approximately up to 180 degrees about a first axis and approximately up to 90 degrees around a second axis, the first axis perpendicular to the second axis,
the plurality of distance sensors being configured to sense a distance between each corresponding panels and a treatment surface; and
the plurality of panels being configured to be arranged into a use position based on the distance between each corresponding panel and the treatment surface.

27. The illuminator of claim 26, wherein the plurality of panels are configured to be disposed in a U-shaped configuration such that the outermost panels of the plurality of panels at least partially face each other.

28. The illuminator of claim 26, further comprising a plurality of cooling fans.
